SEQUENCE LISTING (23 sequences)

SEQ ID NO: 1 - protein, 119 aa, synthetic construct
  REGION 1..119: note = Synthetic sequence
EVQLVETGGGVVKPGGSLRLSCAASGFTFSDYYMSWIRQAPGKGLEWVSYISSSGSTIYYADSVKGRFTISRDNSKNTLYLQMNSLRAEDTAVYYCARESGYDYVFDYWGQGTLVAVSS

SEQ ID NO: 2 - protein, 108 aa, synthetic construct
  REGION 1..108: note = Synthetic sequence
DIQMTQSPSTLSAFVGDRVTITCRASQSISSWLAWYQQKPGKAPKLLIYAASTLQSGVPSRFSGSGSGTEFTLTISSLQPEDFATYYCQQLNSYPITFGQGTRLEIKR

SEQ ID NO: 3 - DNA, 357 bp, synthetic construct
  misc_feature 1..357: note = Synthetic sequence
  source 1..357: mol_type = other DNA
gaggtgcagctggtggagacaggaggaggagtggtgaagccaggaggcagcctgagactgtcctgcgcagcatctggcttcacattttccgactactatatgtcttggatcaggcaggcaccaggcaagggcctggagtgggtgagctacatcagctcctctggctccaccatctactatgccgactctgtgaagggccgcttcacaatcagccgggataactccaagaataccctgtacctgcagatgaacagcctgagagccgaggataccgccgtgtactattgcgccagggagtccggctacgactacgtgttcgattattggggccagggcacactggtggccgtgagctcc

SEQ ID NO: 4 - DNA, 324 bp, synthetic construct
  misc_feature 1..324: note = Synthetic sequence
  source 1..324: mol_type = other DNA
gacatccagatgacacagagcccttccaccctgagcgccttcgtgggcgatcgcgtgacaatcacctgtcgggcctctcagagcatctctagctggctggcctggtaccagcagaagccaggcaaggcccccaagctgctgatctatgccgcctctacactgcagagcggcgtgccatccaggttctccggatctggaagcggaaccgagtttacactgaccatctcctctctgcagcccgaggacttcgccacatactattgccagcagctgaatagctaccctatcacatttggccagggcaccagactggagatcaagagg

SEQ ID NO: 5 - protein, 15 aa, synthetic construct
  REGION 1..15: note = Synthetic sequence
GGGGSGGGGSGGGGS

SEQ ID NO: 6 - protein, 39 aa, Homo sapiens
IEVMYPPPYLDNEKSNGTIIHVKGKHLCPSPLFPGPSKP

SEQ ID NO: 7 - protein, 27 aa, Homo sapiens
FWVLVVVGGVLACYSLLVTVAFIIFWV

SEQ ID NO: 8 - protein, 39 aa, Homo sapiens
RSKRSRLLHSDYMNMTPRRPGPTRKHYQPYAPPRDFAAY

SEQ ID NO: 9 - protein, 42 aa, Homo sapiens
KRGRKKLLYIFKQPFMRPVQTTQEEDGCSCRFPEEEEGGCEL

SEQ ID NO: 10 - protein, 112 aa, Homo sapiens
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 21 aa, Homo sapiens
METDTLLLWVLLLWVPGSTGD

SEQ ID NO: 12 - protein, 113 aa, synthetic construct
  REGION 1..113: note = Synthetic sequence
DILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELK

SEQ ID NO: 13 - protein, 113 aa, synthetic construct
  REGION 1..113: note = Synthetic sequence
EVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSS

SEQ ID NO: 14 - protein, 18 aa, synthetic construct
  REGION 1..18: note = Synthetic sequence
GSTSGSGKPGSGEGSTKG

SEQ ID NO: 15 - protein, 113 aa, synthetic construct
  REGION 1..113: note = Synthetic sequence
EVQLVESGGGLVQPGGSLRLSCAASGFTFSNYGMSWVRQAPGKGLEWIGSIYYSGSTNYNPSLKSLVTISRDNSKNTLYLQMNSLRAEDTATYYCARESIDYWGQGTLVTVSS

SEQ ID NO: 16 - protein, 8 aa, synthetic construct
  REGION 1..8: note = Synthetic sequence
GFTFSNYG

SEQ ID NO: 17 - protein, 7 aa, synthetic construct
  REGION 1..7: note = Synthetic sequence
IYYSGST

SEQ ID NO: 18 - protein, 7 aa, synthetic construct
  REGION 1..7: note = Synthetic sequence
ARESIDY

SEQ ID NO: 19 - protein, 18 aa, synthetic construct
  REGION 1..18: note = Synthetic sequence
GSTSGSGKPGSGEGSTKG

SEQ ID NO: 20 - protein, 19 aa, synthetic construct
  REGION 1..19: note = Synthetic sequence
ATNFSLLKQAGDVEENPGP

SEQ ID NO: 21 - protein, 375 aa, synthetic construct
  REGION 1..375: note = Synthetic sequence
DILLTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELKGSTSGSGKPGSGEGSTKGEVKLQQSGPSLVEPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSSGSTSGSGKPGSGEGSTKGEVQLVESGGGLVQPGGSLRLSCAASGFTFSNYGMSWVRQAPGKGLEWIGSIYYSGSTNYNPSLKSLVTISRDNSKNTLYLQMNSLRAEDTATYYCARESIDYWGQGTLVTVSS

SEQ ID NO: 22 - protein, 480 aa, synthetic construct
  REGION 1..480: note = Synthetic sequence
MALPVTALLLPLALLLHAARPDIQMTQSPSTLSAFVGDRVTITCRASQSISSWLAWYQQKPGKAPKLLIYAASTLQSGVPSRFSGSGSGTEFTLTISSLQPEDFATYYCQQLNSYPITFGQGTRLEIKRGGGGSGGGGSGGGGSEVQLVETGGGVVKPGGSLRLSCAASGFTFSDYYMSWIRQAPGKGLEWVSYISSSGSTIYYADSVKGRFTISRDNSKNTLYLQMNSLRAEDTAVYYCARESGYDYVFDYWGQGTLVAVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 2703 bp, synthetic construct
  misc_feature 1..2703: note = Synthetic sequence
  source 1..2703: mol_type = other DNA
atggccctgccagtgaccgccctgctgctgccactggccctgctgctgcacgcagcccgccctgacatccagatgacacagagcccttccaccctgagcgccttcgtgggcgatcgcgtgacaatcacctgtcgggcctctcagagcatctctagctggctggcctggtaccagcagaagccaggcaaggcccccaagctgctgatctatgccgcctctacactgcagagcggcgtgccatccaggttctccggatctggaagcggaaccgagtttacactgaccatctcctctctgcagcccgaggacttcgccacatactattgccagcagctgaatagctaccctatcacatttggccagggcaccagactggagatcaagaggggaggaggaggaagcggcggaggaggctccggcggcggcggctctgaggtgcagctggtggagacaggaggaggagtggtgaagccaggaggcagcctgagactgtcctgcgcagcatctggcttcacattttccgactactatatgtcttggatcaggcaggcaccaggcaagggcctggagtgggtgagctacatcagctcctctggctccaccatctactatgccgactctgtgaagggccgcttcacaatcagccgggataactccaagaataccctgtacctgcagatgaacagcctgagagccgaggataccgccgtgtactattgcgccagggagtccggctacgactacgtgttcgattattggggccagggcacactggtggccgtgagctccattgaagttatgtatcctcctccttacctagacaatgagaagagcaatggaaccattatccatgtgaaagggaaacacctttgtccaagtcccctatttcccggaccttctaagcccttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatagagtgaagttttccaggtctgccgatgcaccagcatacaagcagggacagaaccagctgtataacgagctgaatctgggccggagagaggagtacgacgtgctggataagaggcgcggcagagatcctgagatgggcggcaagccccggagaaagaaccctcaggagggcctgtacaatgagctgcagaaggacaagatggccgaggcctattctgagatcggcatgaagggagagaggcgccggggcaagggacacgatggcctgtaccagggcctgtccacagccaccaaggacacctatgatgcactgcacatgcaggccctgccaccaagggctactaacttttctcttctcaaacaagctggagatgttgaagaaaatcctggacctatggaaaccgatactctgctcctttgggtgctcctgctgtgggtgcccggcagcaccggcgatgacatcttgctcacacaaactcccttgtccctgcccgtcagtcttggagaccaagcttccatatcttgtagaagttctcaaagtcttgtacaccgaaatggaaatacatacttgcactggtatctccaaaagcccggtcaatcacccaagttgctcatacacaaggtctctaatcggttctctggagtccccgaccgtttcagtggatcaggaagtggaacagacttcacactcaagatatcaagagtcgaggcagaggacctcggggtttacttctgttcccaatcaacacacgtcccacccttgacattcggagccggaactaagttggagttgaaggggtcaacatctggatcaggtaaaccaggtagcggtgaaggaagcactaaaggtgaggtcaagcttcaacaaagtggtccctctcttgtcgagcctggggcttccgttatgatctcctgtaaggcatccggaagttctttcactggatacaatatgaattgggttcgtcaaaatatcgggaagtctttggagtggataggagctatcgacccttactacggcggtacatcctacaatcaaaagttcaagggacgtgccactttgactgtcgacaagtcttcctctactgcttacatgcaccttaagtcacttacttcagaggactctgctgtttactactgtgtcagtgggatggagtactggggtcaagggacttccgtaacagtctcttcagggtcaacttctggttcaggaaaaccaggaagcggggaaggtagtactaaaggagaggtccaattggttgagtctggaggagggctcgtccaaccaggtggaagtttgagactttcatgtgccgcttccggattcacattctccaattacggtatgtcttgggtacgacaagctcccggtaagggactcgagtggatcgggtccatatactactctgggtccacaaattacaatccatccctcaagtcacttgttacaatctcccgggacaattcaaagaatactttgtacttgcaaatgaattctcttcgggcagaggacactgctacttactactgtgcacgagagtctatagactactggggacaagggacgttggtaactgtctccagtcaccatcaccatcaccat

(19) United States
(12) Patent Application Publication
(10) Pub. No.: US 2025/0032543 A1
(43) Pub. Date: Jan. 30, 2025

(54) DUAL TARGETING OF PEDIATRIC MALIGNANCIES THROUGH CAR T-CELLS SECRETING BISPECIFIC INNATE IMMUNE CELL ENGAGERS (BICES)

(71) Applicant: THE CHILDREN'S HOSPITAL OF PHILADELPHIA, Philadelphia, PA (US)

(72) Inventors: Kristopher R. BOSSE, Philadelphia, PA (US); Guillem PASCUAL-PASTO, Philadelphia, PA (US)

(73) Assignee: THE CHILDREN'S HOSPITAL OF PHILADELPHIA, Philadelphia, PA (US)

(21) Appl. No.: 18/716,387

(22) PCT Filed: Dec. 5, 2022
(86) PCT No.: PCT/US2022/080935

§ 371 Date: Jun. 4, 2024

Related U.S. Application Data
(60) Provisional application No. 63/286,130, filed on Dec. 6, 2021.

(51) Int. Cl. A61K 35/17; A61K 39/00; A61P 35/00; C07K 14/725; C07K 16/28; C07K 16/30; C12N 5/0783; C12N 15/86

(52) U.S. Cl. CPC A61K 35/17; A61K 39/4611; A61K 39/4631; A61K 39/4633; A61K 39/464402; A61P 35/00; C07K 14/7051; C07K 16/283; C07K 16/303; C07K 16/3084; C12N 5/0636; C12N 15/86; A61K 2239/13; A61K 2239/21; C07K 2317/31; C07K 2317/565; C07K 2317/569; C07K 2317/622; C07K 2317/732; C07K 2319/03; C07K 2319/21; C12N 2510/00; C12N 2740/15043

(57) ABSTRACT

Provided herein are compositions and methods for providing long-term and sustained CAR T cell efficacy. For example, expression cassettes that encode both a GPC2-targeting chimeric antigen receptor and a bispecific innate immune cell engager binding both GD2 on tumor cells and CD16A on natural killers (NK) and macrophages are provided. In addition, T cells that both expression a GPC2-targeting chimeric antigen receptor on their surface and secrete a bispecific innate immune cell engager binding both GD2 on tumor cells and CD16A on natural killers (NK) and macrophages are provided.

### PRIORITY CLAIM

The application claims benefit of priority to U.S. Provisional Application Ser. No. 63/286,130, filed Dec. 6, 2021, the entire contents of which are hereby incorporated by reference.

### STATEMENT REGARDING FEDERALLY SPONSORED RESEARCH

This invention was made with government support under grant number CA230223 awarded by the National Institutes of Health. The government has certain rights in the invention.

### REFERENCE TO A SEQUENCE LISTING

The instant application contains a Sequence Listing, which has been submitted in ASCII format via EFS-Web and is hereby incorporated by reference in its entirety. Said ASCII copy, created on Dec. 5, 2022, is named CHOPP0050W0_ST26.txt and is 32 kB bytes in size.

### BACKGROUND

### 1. Field

The present invention relates generally to the fields of medicine, oncology, and immunotherapeutics. More particularly, it concerns the combined use of anti-tumor CAR molecules and bispecific innate immune cell engagers (BICE) to activate host innate immune effector cells.

### 2. Description of Related Art

Children with high-risk neuroblastoma have a poor prognosis despite intensive multimodal chemoradiotherapy. Chimeric antigen receptor (CAR) T-cell efficacy in pediatric solid tumors is limited by both the heterogeneous expression of targeted surface antigens and the presence of an immunosuppressive tumor microenvironment. In fact, neuroblastoma cells overcome GPC2 CAR T-cell killing by down-regulating GPC2. As such, methods to circumvent these critical barriers to long-term and sustained CAR T cell efficacy are needed.

### SUMMARY

In one embodiment, provided herein are polynucleotides having a first coding sequence that encodes a chimeric antigen receptor (CAR) comprising, from 5′ to 3′: (i) an ectodomain comprising a single chain antibody variable region that binds selectively to Glypican 2 (GPC2), (ii) a transmembrane domain, and (iii) an endodomain, wherein the endodomain comprises a signal transduction function when the single-chain antibody variable region is engaged with Glypican 2; and a second coding sequence that encodes a fusion protein comprising: (i) a single chain antibody variable region that binds selectively to GD2; and (ii) a single domain antibody (sdAb) that binds CD16A.

In some aspects, the CAR comprises a flexible hinge positioned between the ectodomain and the transmembrane domain. The flexible hinge may be a CD28 hinge having the sequence of SEQ ID NO: 6. In some aspects, the transmembrane domain of the CAR is a CD28 transmembrane domain having the sequence of SEQ ID NO: 7. In some aspects, the endodomain of the CAR comprises a CD28 co-stimulatory domain having the sequence of SEQ ID NO: 8. In some aspects, the endodomain of the CAR comprises a 4-IBB co-stimulatory domain having the sequence of SEQ ID NO: 9. In some aspects, the endodomain of the CAR comprises a CD3zeta co-stimulatory domain having the sequence of SEQ ID NO: 10.

In some aspects, the GPC2 single chain antibody is encoded by the heavy and light chain variable sequences of SEQ ID NOS: 3 and 4, respectively. In some aspects, the GPC2 single chain antibody is encoded by heavy and light chain variable sequences having at least 70%, 80%, 90%, 91%, 92%, 93%, 94%, 95%, 96%, 97%, 98%, or 99% identity to SEQ ID NOS: 3 and 4, respectively. In some aspects, the GPC2 single chain antibody comprises the heavy and light chain variable sequences of SEQ ID NOS: 1 and 2, respectively. In some aspects, the GPC2 single chain antibody comprises heavy and light chain variable sequences having at least 90%, 91%, 92%, 93%, 94%, 95%, 96%, 97%, 98%, or 99% identity to SEQ ID NOS: 1 and 2, respectively.

In some aspects, the CAR has a polypeptide sequence of SEQ ID NO: 22. In some aspects, the CAR has a polypeptide sequence having at least 90%, 91%, 92%, 93%, 94%, 95%, 96%, 97%, 98%, or 99% identity to SEQ ID NO: 22. In some aspects, the CAR has a polypeptide sequence of amino acids 21-480 of SEQ ID NO: 22. In some aspects, the CAR has a polypeptide sequence having at least 90%, 91%, 92%, 93%, 94%, 95%, 96%, 97%, 98%, or 99% identity to amino acids 21-480 of SEQ ID NO: 22.

In some aspects, the second coding region is a bipecific innate immune cell engager (BiCE) that comprises a GD2 single chain antibody variable region fused to a CD16A single domain antibody. In some aspects, the GD2 single chain antibody comprises the heavy and light chain variable sequences of SEQ ID NOS: 12 and 13, respectively. In some aspects, the GD2 single chain antibody comprises heavy and light chain variable sequences having at least 90%, 91%, 92%, 93%, 94%, 95%, 96%, 97%, 98%, or 99% identity to SEQ ID NOS: 12 and 13, respectively.

In some aspects, the CD16A single domain antibody is characterized by CDR sequences SEQ ID NOS: 16-18. In some aspects, the CD16A single domain antibody comprises the sequence of SEQ ID NO: 15. In some aspects, the CD16A single domain antibody comprises a sequence having at least 90%, 91%, 92%, 93%, 94%, 95%, 96%, 97%, 98%, or 99% identity to SEQ ID NO: 15.

In some aspects, the BiCE has a polypeptide sequence of SEQ ID NO: 21. In some aspects, the BiCE has a polypeptide sequence having at least 90%, 91%, 92%, 93%, 94%, 95%, 96%, 97%, 98%, or 99% identity to SEQ ID NO: 21.

In some aspects, the polynucleotides further comprise a sequence encoding a CD8 leader sequence positioned 5′ of the first coding sequence.

In some aspects, the polynucleotides further comprise a sequence encoding a cleavable peptide positioned between the first coding sequence and the second coding sequence. The cleavable peptide may be P2A.

In some aspects, the polynucleotides further comprise a sequence encoding a Igκ leader sequence positioned 5′ of the second coding sequence.

In some aspects, the polynucleotides further comprise a His6 sequence positioned 3′ of the second coding sequence.

In some aspects, the polynucleotides further comprise a promoter sequence positioned 5′ of the first coding sequence. The promoter may be a constitutive promoter. The promoter may be an EF1α promoter.

In some aspects, the polynucleotides have a sequence of SEQ ID NO: 23. In some aspects, the polynucleotides have a sequence having at least 70%, 80%, 90%, 91%, 92%, 93%, 94%, 95%, 96%, 97%, 98%, or 99% identity to SEQ ID NO: 23. In some aspects, the polynucleotides have a sequence of nucleotides 61-2685 of SEQ ID NO: 23. In some aspects, the polynucleotides have a sequence having at least 70%, 80%, 90%, 91%, 92%, 93%, 94%, 95%, 96%, 97%, 98%, or 99% identity to nucleotides 61-2685 of SEQ ID NO: 23.

In one embodiment, provided herein are expression vectors comprising the polynucleotide of any one of the present embodiments.

In one embodiment, provided herein are viral vectors comprising the polynucleotide of any one of the present embodiments. The viral vector may be a lentiviral vector.

In one embodiment, provided herein are cells comprising the polynucleotide of any one of the present embodiments. The polynucleotide may be integrated into the genome of the cell. The cell may be a T cell, which may express the chimeric antigen receptor on its surface and secret the fusion protein.

In one embodiment, provided herein are compositions comprising the cells of any one of the present embodiments, the fusion protein encoded by the second coding sequence, and a pharmaceutically acceptable carrier. The compositions may further comprise an additional active agent.

In one embodiment, provided herein are methods of treating cancer in a patient in need thereof, the methods comprising administering to the patient an effective amount of the cells of any one of the present embodiments or the composition of any one of the present embodiments. The cancer may be a solid tumor. The cancer may be a neuroblastoma or glioma. The patient may be a pediatric patient. The cells of the cancer may express GPC2 on their surface. The cells of the may cancer express GD2 on their surface. The methods may activate the patient's innate immune effector cells to target the cancer. The methods may induce antibody-dependent cellular cytotoxicity against the cancer. The methods may induce antibody-dependent cellular phagocytosis against the cancer. The cells may be allogeneic or autologous to the patient. The cells or the composition may be administered systemically. The methods may further comprise administering a second anti-cancer therapy to the patient, such as, for example, a surgical therapy, chemotherapy, radiation therapy, cryotherapy, hormone therapy, immunotherapy, or cytokine therapy.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following drawings form part of the present specification and are included to further demonstrate certain aspects of the present invention. The invention may be better understood by reference to one or more of these drawings in combination with the detailed description of specific embodiments presented herein.

FIGS. 1A-E. Neuroblastoma cells overcome GPC2 CAR T-cell killing by down-regulating GPC2, but upregulate NK cell ligands. (FIG. 1A) Neuroblastoma cell viability after GPC2 or CD19 CAR T-cell treatment in vitro for 4 days [effector:tumor (E:T) ratio of 1:2.5]. Relative cell surface expression of GPC2 (FIG. 1B), GD2 (FIG. 1C), MICA/B (FIG. 1D) and ULBP-1 (FIG. 1E) in the residual, CD45-negative neuroblastoma cells after CAR T-cell exposure for 4 days. Individual replicates (n=3) with means and standard deviations are shown. P values are indicated in Figure. EBC1=NB−EbC1.

FIGS. 2A-B. Engineering CAR T-cells to express GPC2 CAR in the cell membrane and to secrete bispecific innate immune cell engagers (BICEs). (FIG. 2A) Graphical abstract summarizing the approach of CAR T-cells secreting BiCEs. BiCEs are composed of tumor-targeted GD2 single-chain variable fragments (scFv) linked to single-domain antibodies (sdAb) targeting CD16A in innate immune cells including natural killer (NK) cells or macrophages. (FIG. 2B) Bicistronic lentiviral vector design. Bicistronic vectors lead to i) GPC2 CAR expression on the cell surface, ii) CD28-based intracellular stimulatory signaling and iii) secretion of GD2-targeted BiCEs. An additional GPC2 CAR secreting CD19-directed BiCEs was developed as control for non-relevant targeting of BiCEs. BiCEs were tagged with His-tag to facilitate their detection in vitro and in vivo. First-generation GPC2.CD28 and CD19.4IBB CARs were also generated.

FIGS. 3A-E. Production and binding characterization of BICEs. (FIG. 3A) Flow cytometry histograms showing His-tag cell surface expression in neuroblastoma cells incubated with concentrated supernatants (cSNs) from CAR constructs (CAR.GPC2, CAR.GPC2-BiCE.GD2 or CAR.GPC2-BICE.CD19) or 1×PBS and then stained with phycoerythrin (PE)-tagged anti-His-tag antibody. Neuroblastoma cells with high (NB-EBC1 and SMS-SAN) and low (SY5Y) GD2 expression were selected. Leukemia (NALM6) cells with high CD19 were utilized as controls. (FIG. 3B) Concentration-dependent binding of CAR.GPC2-BICE.GD2 cSN in GD2-high NB-EBC1 cells. (FIG. 3C) Binding competition assay between FDA-approved anti-GD2 antibody dinutuximab and CAR.GPC2-BiCE.GD2 cSNs. (FIG. 3D) Graphical scheme of the “sandwich” binding assays where GD2-expressing neuroblastoma cells were incubated with bicistronic vector cSNs and then stained with recombinant human (rh) CD16A protein previously conjugated with APC fluorophore. (FIG. 3E) Flow cytometry histograms showing CD16A sdAb APC staining in GD2-positive cells incubated with GPC2.CAR-GD2.BiCE cSN but not with control vector cSN (GPC2.CAR-CD19.BiCE).

FIGS. 4A-G. Antibody-dependent cellular cytotoxicity (ADCC) and phagocytosis (ADCP) of GD2-targeted bispecific innate immune cell engagers (BiCEs). (FIG. 4A) Luciferase-labeled neuroblastoma cell lines with high (NB-EBC1 and SMS-SAN) or low (SY5Y) GD2 expression were exposed to cSN from CAR constructs together with human primary natural killer (NK) cells in a NK:tumor ratio of 10:1 for 24 h. FDA-approved dinutuximab was used as positive control. Specific tumor lysis was determined measuring the luciferase tumor signal. (FIG. 4B) Specific lysis of luciferase-labelled NB-EBC1 cells exposed to different concentrations of GD2.BiCEs (measured by His-tag ELISA) and two different human primary NK:tumor ratios for 24 h. (FIG. 4C) Specific lysis of luciferase-labelled NB-EBC1 cells exposed to different cSN from CAR constructs together with CD16a isogenic or wild-type NK92 cells in a NK:tumor ratio of 10:1 for 24 h. (FIG. 4D) NB-EBC1 cells were exposed to cSN from BiCE constructs (GD2 and CD19) together with primary NK cells from 3 different donors and NK cells analyzed for activation by co-staining with CD69 and CD107a by flow cytometry and (FIG. 4E) quantifying secretion of IFN-γ by ELISA. (FIG. 4F) GFP-labeled NB-EBC1 cells were exposed to cSN from CAR constructs together with human monocyte-differentiated macrophages. Phagocytosis was measured by quantifying GFP/CD11b-positive macrophages by flow cytometry as shown. FDA-approved dinutuximab was used as positive control. (FIG. 4G) Quantification of phagocytosis in FIG. 4F. Means and SDs are represented.

FIGS. 5A-C. Human primary T-cells transduced with bicistronic vectors express GPC2 CAR on the surface that induces GPC2-dependent tumor killing and T-cell activation. (FIG. 5A) Surface expression of GPC2 CAR in T cells transduced with first-generation and bicistronic vectors at the end of T-cell expansion (day 14). Recombinant human GPC2 protein tagged with PE was used to measure CAR expression by flow cytometry. Percentage of CAR positive cells is indicated. (FIG. 5B) T-cell killing assay of the different CAR vectors against GPC2-expressing luciferase-labelled neuroblastoma and high-grade glioma (HGG) cells at different T-cell:tumor ratios for 24 h. Specific lysis was determined measuring luciferase tumor signal. (FIG. 5C) Secretion of IFNγ by the different CAR constructs in the presence of GPC2-high or -low target cells at 24 h post co-culture with a 2.5:1 T-cell:tumor ratio.

FIGS. 6A-E. Human primary T-cells transduced with bicistronic vectors secret BiCEs and activate primary NK cells to kill GD2-expressing cells. (FIG. 6A) Secretion of BiCEs measured by ELISA in GPC2.CAR-GD2.BiCE and GD2.BiCE transduced T-cells alone, activated with CD3/CD28 stimulatory beads (aT-cell) or exposed to neuroblastoma cells with different amounts of GPC2 (n=6 cell lines). (FIG. 6B) NK-mediated specific lysis of luciferase-tagged NB-EBC1 exposed to different dilutions of T-cell-secreted BiCEs (1:8, 1:16 and 1:32) from different constructs together with human primary NK cells at a 10:1 NK:tumor ratio for 24 h. (FIG. 6C) NK-mediated specific lysis of luciferase-tagged 7316-3058 high grade glioma cells exposed to T-cell-secreted BiCEs (1:8 dilution) from different constructs together with human primary NK cells at a 10:1 NK:tumor ratio for 24 h. FDA-approved anti-GD2 dinutuximab was used as positive control for NK cell killing. (FIG. 6D) Schematic illustration of Transwell assays to evaluate bystander NK cell activation mediated by T-cell-secreted GD2 BiCEs. (FIG. 6E) Quantification of tumor cell viability (% of CD19.CAR T-cell-treated controls) in both top and bottom Transwell chambers. *P<0.0001 (Sidak's multiple comparisons test; GPC2.CAR vs GPC2.CAR-GD2.BiCE).

FIGS. 7A-E. Intravenous injection of T-cells transduced with bicistronic vectors locally release GD2 BiCEs to enhance accumulation of NK cells in the tumor bed. (FIG. 7A) Schematic in vivo protocol for the biodistribution/pharmacokinetic study of GD2 BiCEs compared to FDA-approved anti-GD2 monoclonal antibody (mAb) dinutuximab in mice. (FIG. 7B) GD2 BiCE (in blue; pg/mL) and GD2 mAb concentrations (in green; ng/ml) in tumors and mouse healthy tissues (adjacent muscle, liver, spleen, lung, heart, brainstem, cerebellum and cortex) harvested at day 5-7 after the first dose of either T-cells or dinutuximab. ***P<0.0001, *P=0.01, ##P=0.002 and ####P<0.0001. (*; tumor vs normal, #; normal vs tumor). (FIG. 7C) IHC staining of human CD3 (human T-cells) in neuroblastoma PDX tumors isolated from biodistribution assay [dinutuximab-treated (left) and GPC2.CAR-GD2.BiCE T-cell-treated (right)]. Scale bars represent 1 mm. (FIG. 7D) Schematic in vivo protocol for the pharmacodynamics/NK-cell tumor accumulation study. (FIG. 7E) Follow-up of intratumor NK92-cell retention in vivo by IVIS imaging of mice bearing neuroblastoma PDXs (left). Quantification of NK92-cell retention in both GPC2.CAR-GD2.BiCE or GPC2.CAR-CD19.BiCE-treated animals measuring AUC3-96 h luciferase signal (right). *P=0.015 (Mann-Whitney t-test). AUC=area under the curve.

FIGS. 8A-G. GPC2.CAR-GD2.BICE T cells have improved in vivo efficacy compared to GPC2 CAR T cells alone when administered with donor-matched PBMCs. (FIG. 8A) Flow cytometry histograms showing GPC2 expression (left) and GPC2 cell surface molecules (right) in neuroblastoma COG-N-421x, COG-N-561x and COG-N-603x PDX models (n=3 different tumors). (FIG. 8B) Flow cytometry histograms showing GD2 expression (left) and GD2 cell surface molecules (right) in neuroblastoma COG-N-421x, COG-N-561x and COG-N-603x PDX models (n=3 different tumors). (FIG. 8C) Schematic representation for the in vivo protocol used for efficacy studies. (FIG. 8D) CD16a-APC and CD3/CD19-PE flow cytometry expression in both regular peripheral blood mononuclear cells (PBMCs) and PBMCs depleted for T-cells and B-cells [(en) PBMCs]. CAR T-cell-donor matched (en) PBMCs were utilized as innate immune effector cells in efficacy studies. (FIG. 8E) COG-N-421x tumor growth curves (left), tumor volume at day 14 (****P<0.0001; middle) and progression-free survival (PFS; right) of mice, in which experimental endpoint was when tumor volume reached 2.00 cm³. (FIG. 8F) High tumor burden COG-N-561x tumor growth curves (left), tumor volume at day 14 (*P-0.04 and ***P=0.0002; middle) and PFS of mice (right). (FIG. 8G) High tumor burden COG-N-603x tumor growth curves (left), tumor volume at day 14 (*P=0.04 and ***P=0.0002; middle) and PFS (right) of mice. Means and SEM are represented. Each treatment group contained n=5-7 animals.

### DETAILED DESCRIPTION

Chimeric antigen receptor (CAR) T-cell efficacy in pediatric solid tumors is limited by both the heterogeneous expression of targeted surface antigens and the presence of an immunosuppressive tumor microenvironment. To circumvent these critical barriers to long-term and sustained CAR T cell efficacy, bicistronic constructs are provided that enable 1) T-cell expression of a CAR molecule directed towards GPC2, a cell surface oncoprotein expressed in a variety of pediatric malignancies, and 2) secretion of a bispecific innate immune cell engager (BICE) binding both GD2 on tumor cells and CD16A on natural killers (NK) and macrophages. The addition of a BICE into a GPC2 CAR construct allows the adoptively transferred T cells to activate the host innate immune effector cells to provide an additional antitumor effect. The CAR region is composed by a single-chain variable fragment (scFv) targeting GPC2 (D3 binder; PCT Publn. WO2017/083296, which is incorporated by reference herein in its entirety) preceded by a CD8 leader sequence and followed by CD28 hinge/transmembrane/co-stimulatory domains, and a CD3 zeta co-stimulatory domain. The BICE sequence is preceded by an Ig Kappa leader motif to enhance secretion and followed by a His-tag element to detect the product in vitro and in vivo. The BICE transgene is composed of a GD2 scFv linked to a CD16A single-domain antibody (sdAb; PCT Publn. WO2018/039626, which is incorporated by reference herein in its entirety). The entire bicistronic construct was placed under the control of the constitutive EF1alpha promoter. The bicistronic construct is efficiently transferred into primary human T cells using lentiviral vectors. In vitro, transduced GPC2.CAR-GD2.BICE T cells induced GPC2-dependent killing of neuroblastoma and high-grade glioma (HGG) tumor cells in co-culture assays. Concentrated supernatants from GPC2.CAR-GD2.BICE T cells induced antibody-dependent cellular cytotoxicity and phagocytosis (ADCC and ADCP, respectively) when added to neuroblastoma and HGG cells in the presence of NKs and macrophages. In mice bearing neuroblastoma patient-derived xenografts (PDXs), intravenous injection of T cells transduced with bicistronic vectors locally delivered GD2 BICEs in the tumor bed but not in healthy tissues and promoted intratumor accumulation of luciferase-labelled CD16-overexpressing NK92 cells. Most importantly, GPC2.CAR-GD2.BICE T-cells strongly controlled tumor growth of mice bearing diverse neuroblastoma PDXs expressing different levels of GPC2 and humanized donor-matched innate immune cells, and such efficacy is superior to first-generation GPC2.CAR T cells alone.

### I. GPC2 Chimeric Antigen Receptors

Chimeric antigen receptor (CAR) molecules are recombinant fusion proteins and are distinguished by their ability to both bind antigen and transduce activation signals via immunoreceptor activation motifs (ITAMs) present in their cytoplasmic tails in order to activate genetically modified immune effector cells for killing, proliferation, and cytokine production. Receptor constructs utilizing an antigen-binding moiety (for example, generated from single chain antibodies (scFv)) afford the additional advantage of being “universal” in that they bind native antigen on the target cell surface in an HLA-independent fashion.

Embodiments of the CARs described herein include nucleic acids encoding an antigen-specific CAR polypeptide comprising an intracellular signaling domain, a transmembrane domain, and an extracellular domain comprising an antigen-binding domain. Optionally, a CAR can comprise a hinge domain positioned between the transmembrane domain and the antigen binding domain. A CAR may further comprise a signal peptide that directs expression of the CAR to the cell surface. One embodiment includes a chimeric antigen receptor comprising (i) an ectodomain comprising single chain antibody variable region that binds selectively to Glypican 2, wherein said antibody: (a) is an IgG antibody; (b) inhibits cancer cell growth; (c) induces cancer cell death, and has a flexible hinge attached at the C-terminus of said single chain antibody variable region; (ii) a transmembrane domain; and (iii) an endodomain, wherein said endodomain comprises a signal transduction function when said single-chain antibody variable region is engaged with Glypican 2. The transmembrane and endodomains may be derived from the same molecule. The endodomain may comprise a CD3-zeta domain or a high affinity FcsRI. The flexible hinge may be from CD8a or Ig. Still another embodiment comprises a cell expressing the chimeric antigen receptor as defined above. For example, a CAR may comprise a signal peptide from CD8. In one embodiment, the CAR comprises a single-chain variable fragment (scFv) targeting GPC2 (D3 binder; PCT Publn. WO2017/083296, which is incorporated by reference herein in its entirety) preceded by a CD8 leader sequence and followed by CD28 hinge/transmembrane/co-stimulatory domains, and a CD3 zeta co-stimulatory domain. A CAR may also be co-expressed with a membrane-bound cytokine to improve persistence. For example, a CAR may be co-expressed with membrane-bound IL-15.

A CAR consisting of the present disclosure may have a sequence as provided in Table 1.

TABLE 1
GPC2-targeting CAR

| | | SEQ |
|---|---|---|
| | | ID |
| Region | Sequence | NO: |
| CAR | MALPVTALLLPLALLLHAARPDIQMTQSPSTLSAFVGDRVT | 22 |
| | ITCRASQSISSWLAWYQQKPGKAPKLLIYAASTLQSGVPSR | |
| | FSGSGSGTEFTLTISSLQPEDFATYYCQQLNSYPITFGQGT | |
| | RLEIKRGGGGSGGGGSGGGGSEVQLVETGGGVVKPGGSLRL | |
| | SCAASGFTFSDYYMSWIRQAPGKGLEWVSYISSSGSTIYYA | |
| | DSVKGRFTISRDNSKNTLYLQMNSLRAEDTAVYYCARESGY | |
| | DYVFDYWGQGTLVAVSSIEVMYPPPYLDNEKSNGTIIHVK | |
| | GKHLCPSPLFPGPSKPFWVLVVVGGVLACYSLLVTVAFIIF | |
| | WVRSKRSRLLHSDYMNMTPRRPGPTRKHYQPYAPPRDFA | |
| | AYRVKFSRSADAPAYKQGQNQLYNELNLGRREEYDVLDK | |
| | RRGRDPEMGGKPRRKNPQEGLYNELQKDKMAEAYSEIGM | |
| | KGERRRGKGHDGLYQGLSTATKDTYDALHMQALPPR | |

Depending on the arrangement of the domains of the CAR and the specific sequences used in the domains, immune effector cells expressing the CAR may have different levels activity against target cells. Different CAR sequences may be introduced into immune effector cells to generate engineered cells, the engineered cells selected for elevated SRC, and the selected cells tested for activity to identify the CAR constructs predicted to have the greatest therapeutic efficacy.

A chimeric antigen receptor can be produced by any means known in the art, though preferably it is produced using recombinant DNA techniques. A nucleic acid sequence encoding the several regions of the chimeric antigen receptor can be prepared and assembled into a complete coding sequence by standard techniques of molecular cloning (genomic library screening, PCR, primer-assisted ligation, scFv libraries from yeast and bacteria, site-directed mutagenesis, etc.). The resulting coding region can be inserted into an expression vector and used to transform a suitable expression host allogeneic or autologous immune effector cells, such as a T cell.

The chimeric construct may be introduced into immune effector cells as naked DNA or in a suitable vector. Methods of stably transfecting cells by electroporation using naked DNA are known in the art. See, e.g., U.S. Pat. No. 6,410,319. Naked DNA generally refers to the DNA encoding a chimeric receptor contained in a plasmid expression vector in proper orientation for expression. Alternatively, a viral vector (e.g., a retroviral vector, adenoviral vector, adeno-associated viral vector, or lentiviral vector) can be used to introduce the chimeric construct into immune effector cells. Suitable vectors for use in accordance with the method of the present invention are non-replicating in the immune effector cells. A large number of vectors are known that are based on viruses, where the copy number of the virus maintained in the cell is low enough to maintain the viability of the cell, such as, for example, vectors based on HIV, SV40, EBV, HSV, or BPV.

### A. Antigen Binding Domains

An antigen binding domain may comprise complementary determining regions of a monoclonal antibody, variable regions of a monoclonal antibody, and/or antigen binding fragments thereof. The antigen binding regions or domains may comprise a fragment of the VH and VL chains of a single-chain variable fragment (scFv) derived from a particular mouse, human, or humanized monoclonal antibody. The fragment can also be any number of different antigen binding domains of an antigen-specific antibody. The fragment may be an antigen-specific scFv encoded by a sequence that is optimized for human codon usage for expression in human cells.

The prototypical CAR encodes a scFv comprising VH and VL domains derived from one monoclonal antibody (mAb), coupled to a transmembrane domain and one or more cytoplasmic signaling domains (e.g. costimulatory domains and signaling domains). Thus, a CAR may comprise the amino acid sequences of the VH and VL domains of mAb D3 (M201) that binds to GPC2, as shown in Table 2 and as encoded by the sequence in Table 3.

TABLE 2
mAb D3 (M201) variable region amino
acid sequences.

| | | SEQ |
|---|---|---|
| | | ID |
| Region | Sequence | NO: |
| VH | EVQLVETGGGVVKPGGSLRLSCAASGFTFSDYYMSWIR | 1 |
| | QAPGKGLEWVSYISSSGSTIYYADSVKGRFTISRDNSK | |
| | NTLYLQMNSLRAEDTAVYYCARESGYDYVFDYWGQGTL | |
| | VAVSS | |
| VL | DIQMTQSPSTLSAFVGDRVTITCRASQSISSWLAWYQQ | 2 |
| | KPGKAPKLLIYAASTLQSGVPSRFSGSGSGTEFTLTIS | |
| | SLQPEDFATYYCQQLNSYPITFGQGTRLEIKR | |

TABLE 3
mAb D3 (M201) variable region nucleotide
sequences.

| | | SEQ |
|---|---|---|
| | | ID |
| Region | Sequence | NO: |
| VH | GAGGTGCAGCTGGTGGAGACAGGAGGAGGAGTGGTGAAG | 3 |
| | CCAGGAGGCAGCCTGAGACTGTCCTGCGCAGCATCTGGC | |
| | TTCACATTTTCCGACTACTATATGTCTTGGATCAGGCAG | |
| | GCACCAGGCAAGGGCCTGGAGTGGGTGAGCTACATCAGC | |
| | TCCTCTGGCTCCACCATCTACTATGCCGACTCTGTGAAG | |
| | GGCCGCTTCACAATCAGCCGGGATAACTCCAAGAATACC | |
| | CTGTACCTGCAGATGAACAGCCTGAGAGCCGAGGATACC | |
| | GCCGTGTACTATTGCGCCAGGGAGTCCGGCTACGACTAC | |
| | GTGTTCGATTATTGGGGCCAGGGCACACTGGTGGCCGTG | |
| | AGCTCC | |
| VL | GACATCCAGATGACACAGAGCCCTTCCACCCTGAGCGCC | 4 |
| | TTCGTGGGCGATCGCGTGACAATCACCTGTCGGGCCTCT | |
| | CAGAGCATCTCTAGCTGGCTGGCCTGGTACCAGCAGAAG | |
| | CCAGGCAAGGCCCCCAAGCTGCTGATCTATGCCGCCTCT | |
| | ACACTGCAGAGCGGCGTGCCATCCAGGTTCTCCGGATCT | |
| | GGAAGCGGAACCGAGTTTACACTGACCATCTCCTCTCTG | |
| | CAGCCCGAGGACTTCGCCACATACTATTGCCAGCAGCTG | |
| | AATAGCTACCCTATCACATTTGGCCAGGGCACCAGACTG | |
| | GAGATCAAGAGG | |

A single chain variable fragment (scFv) is a fusion of the variable regions of the heavy and light chains of immunoglobulins, linked together with a short linker. This chimeric molecule retains the specificity of the original immunoglobulin, despite removal of the constant regions and the introduction of a linker peptide. This modification usually leaves the specificity unaltered. scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine and glycine. However, other residues can function as well. For example, the linker may have a proline residue two residues after the V_{H} C terminus and an abundance of arginines and prolines at other positions. The CAR variable region may contain a (Gly4Ser) 3 linker sequence, as shown in Table 4.

TABLE 4
Linker used in GPC2 CAR variable region

| Region | Sequence | SEQ ID NO: |
|---|---|---|
| Linker | GGGGSGGGGSGGGGS | 5 |

U.S. Pat. No. 5,856,456 provides peptide linkers for use in connecting polypeptide constituents to make fusion proteins, e.g., single chain antibodies. The linker is up to about 50 amino acids in length, contains at least one occurrence of a charged amino acid (preferably arginine or lysine) followed by a proline, and is characterized by greater stability and reduced aggregation.

### B. Hinge Domains

A CAR polypeptide may include a hinge domain positioned between the antigen binding domain and the transmembrane domain. In some cases, a hinge domain may be included in CAR polypeptides to provide adequate distance between the antigen binding domain and the cell surface or to alleviate possible steric hindrance that could adversely affect antigen binding or effector function of CAR-modified immune effector cells. The hinge domain may comprise a sequence that binds to an Fc receptor, such as FcγR2a or FcγR1a. For example, the hinge sequence may comprise an Fc domain from a human immunoglobulin (e.g., IgG1, IgG2, IgG3, IgG4, IgA1, IgA2, IgM, IgD or IgE) that binds to an Fc receptor.

A CAR hinge domain may be derived from human immunoglobulin (Ig) constant region or a portion thereof including the Ig hinge, or from human CD8 α transmembrane domain and CD8a-hinge region. A CAR hinge domain may comprise a hinge-CH₂-CH₃ region of antibody isotype IgG4. The hinge domain (and/or the CAR) may not comprise a wild type human IgG4 CH2 and CH3 sequence. Point mutations may be introduced in antibody heavy chain CH₂ domain to reduce glycosylation and non-specific Fc gamma receptor binding of CAR-modified immune effector cells.

The hinge domain may comprise a sequence that is about 85%, 90%, 91%, 92%, 93%, 94%, 95%, 96%, 97%, 98%, 99% or 100% identical to an IgG4 hinge domain, a CD8a hinge domain, a CD28 hinge domain, or an engineered hinge domain. The CAR polypeptide may contain a CD28 hinge, as shown in Table 5.

TABLE 5
CD28 hinge domain sequence

| Region | Sequence | SEQ ID NO: |
|---|---|---|
| CD28 | IEVMYPPPYLDNEKSNGTIIHVK | 6 |
| hinge | GKHLCPSPLFPGPSKP | |

### C. Transmembrane Domains

The antigen-specific extracellular domain and the intracellular signaling-domain may be linked by a transmembrane domain. Polypeptide sequences that can be used as part of transmembrane domain include, without limitation, the human CD4 transmembrane domain, the human CD28 transmembrane domain, the transmembrane human CD35 domain, a cysteine mutated human CD32 domain, or other transmembrane domains from other human transmembrane signaling proteins, such as CD16, CD8, and erythropoietin receptor. For example, the transmembrane domain may comprise a sequence at least 85%, 90%, 91%, 92%, 93%, 94%, 95%, 96%, 97%, 98%, 99% or 100% identical to one of those provided in U.S. Patent Publication No. 2014/0274909 (e.g. a CD8 and/or a CD28 transmembrane domain) or U.S. Pat. No. 8,906,682 (e.g. a CD8α transmembrane domain), both incorporated herein by reference. Transmembrane regions may be derived from (i.e. comprise at least the transmembrane region(s) of) the alpha, beta or zeta chain of the T-cell receptor, CD3 epsilon, CD45, CD4, CD5, CD8, CD9, CD16, CD22, CD33, CD37, CD64, CD80, CD86, CD134, CD137, CD154. In certain specific aspects, the transmembrane domain can be 85%, 90%, 91%, 92%, 93%, 94%, 95%, 96%, 97%, 98%, 99% or 100% identical to a CD8a transmembrane domain or a CD28 transmembrane domain. The CAR polypeptide may contain a CD28 transmembrane, as shown in Table 6.

TABLE 6
CD28 transmembrane domain sequence

| Region | Sequence | SEQ ID NO: |
|---|---|---|
| CD28 | FWVLVVVGGVLACYSLLVTVAFIIFWV | 7 |
| TM | | |

### D. Intracellular Signaling Domains

The intracellular signaling domain of a CAR is responsible for activation of at least one of the normal effector functions of the immune cell engineered to express the CAR. The term “effector function” refers to a specialized function of a differentiated cell. Effector function of a T cell, for example, may be cytolytic activity or helper activity including the secretion of cytokines. Effector function in a naïve, memory, or memory-type T cell includes antigen-dependent proliferation. Thus the term “intracellular signaling domain” refers to the portion of a protein that transduces the effector function signal and directs the cell to perform a specialized function. The intracellular signaling domain may be derived from the intracellular signaling domain of a native receptor. Examples of such native receptors include the zeta chain of the T-cell receptor or any of its homologs (e.g., eta, delta, gamma, or epsilon), MB1 chain, B29, Fc RIII, Fc RI, and combinations of signaling molecules, such as CD3ζ and CD28, CD27, 4-1BB/CD137, ICOS/CD278, IL-2Rβ/CD122, IL-2Rα/CD132, DAP10, DAP12, CD40, OX40/CD134, and combinations thereof, as well as other similar molecules and fragments. Intracellular signaling portions of other members of the families of activating proteins can be used.

While the entire intracellular signaling domain may be employed, in many cases it will not be necessary to use the entire intracellular polypeptide. To the extent that a truncated portion of the intracellular signaling domain may find use, such truncated portion may be used in place of the intact chain as long as it still transduces the effector function signal. The term “intracellular signaling domain” is thus meant to include a truncated portion of the intracellular signaling domain sufficient to transduce the effector function signal, upon CAR binding to a target. One or multiple cytoplasmic domains may be employed, as so-called third generation CARs have at least two or three signaling domains fused together for additive or synergistic effect, for example the CD28 and 4-1BB can be combined in a CAR construct. In certain specific aspects, the intracellular signaling domain comprises a sequence 85%, 90%, 91%, 92%, 93%, 94%, 95%, 96%, 97%, 98%, 99% or 100% identical to a CD35 intracellular domain, a CD28 intracellular domain, a CD137 intracellular domain, or a domain comprising a CD28 intracellular domain fused to the 4-1BB intracellular domain. The CAR polypeptide may contain a CD28 or 4-1BB intracellular signaling domain fused to a CD35 intracellular signaling domain, as shown in Table 7.

TABLE 7
Sequences of exemplary intracellular
signaling domains.

| | | SEQ |
|---|---|---|
| | | ID |
| Region | Sequence | NO: |
| CD28 | RSKRSRLLHSDYMNMTPRRPGPTRKHYQPYAPPRDF | 8 |
| signaling | AAY | |
| 4-1BB | KRGRKKLLYIFKQPFMRPVQTTQEEDGCSCRFPEEE | 9 |
| signaling | EGGCEL | |
| CD3z | RVKFSRSADAPAYKQGQNQLYNELNLGRREEYDVLD | 10 |
| signaling | KRRGRDPEMGGKPRRKNPQEGLYNELQKDKMAEAYS | |
| | EIGMKGERRRGKGHDGLYQGLSTATKDTYDALHMQA | |
| | LPPR | |

### E. Immune Effector Cells

Immune effectors cells may be T cells (e.g., regulatory T cells, CD4+ T cells, CD8+ T cells, or gamma-delta T cells), natural killer (NK) cells, invariant NK cells, or NKT cells. Also provided herein are methods of producing and engineering the immune effector cells as well as methods of using and administering the cells for adoptive cell therapy, in which case the cells may be autologous or allogeneic. Thus, the immune effector cells may be used as immunotherapy, such as to target cancer cells.

The immune effector cells may be isolated from subjects, particularly human subjects. The immune effector cells can be obtained from a subject of interest, such as a subject suspected of having a particular disease or condition, a subject suspected of having a predisposition to a particular disease or condition, a subject who is undergoing therapy for a particular disease or condition, a subject who is a healthy volunteer or healthy donor, or from a blood bank. Immune effector cells can be collected, enriched, and/or purified from any tissue or organ in which they reside in the subject including, but not limited to, blood, cord blood, spleen, thymus, lymph nodes, bone marrow, tissues removed and/or exposed during surgical procedures, and tissues obtained via biopsy procedures. The isolated immune effector cells may be used directly, or they can be stored for a period of time, such as by freezing.

Tissues/organs from which the immune effector cells are enriched, isolated, and/or purified may be isolated from both living and non-living subjects, wherein the non-living subjects are organ donors. Immune effector cells isolated from cord blood may have enhanced immunomodulation capacity, such as measured by CD4- or CD8-positive T cell suppression. The immune effector cells may be isolated from pooled blood, particularly pooled cord blood, for enhanced immunomodulation capacity. The pooled blood may be from 2 or more sources, such as 3, 4, 5, 6, 7, 8, 9, 10 or more sources (e.g., donor subjects).

The population of immune cells can be obtained from a subject in need of therapy or suffering from a disease associated with reduced immune effector cell activity. Thus, the cells will be autologous to the subject in need of therapy. Alternatively, the population of immune effector cells can be obtained from a donor, preferably an allogeneic donor. Allogeneic donor cells may or may not be human-leukocyte-antigen (HLA)-compatible. To be rendered subject-compatible, allogeneic cells can be treated to reduce immunogenicity.

The immune effector cells may be T cells. The T cells may be derived from the blood, bone marrow, lymph, umbilical cord, or lymphoid organs. The T cells may be human T cells. The T cells typically are primary cells, such as those isolated directly from a subject and/or isolated from a subject and frozen. The cells may include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. For off-the-shelf technologies, the cells may be derived from pluripotent and/or multipotent cells, such as stem cells, such as induced pluripotent stem cells (iPSCs).

Among the sub-types and subpopulations of T cells (e.g., CD4⁺ and/or CD8⁺ T cells) are naive T (T_{N}) cells, effector T cells (T_{EFF}), memory T cells and sub-types thereof, such as stem cell memory T (TSC_{M}), central memory T (TC_{M}), effector memory T (T_{EM}), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

One or more of the T cell populations may be enriched for or depleted of cells that are positive for a specific marker, such as surface markers, or that are negative for a specific marker. In some cases, such markers are those that are absent or expressed at relatively low levels on certain populations of T cells (e.g., non-memory cells) but are present or expressed at relatively higher levels on certain other populations of T cells (e.g., memory cells).

T cells may be separated from a PBMC sample by negative selection of markers expressed on non-T cells, such as B cells, monocytes, or other white blood cells, such as CD14. In some aspects, a CD4⁺ or CD8⁺ selection step is used to separate CD4⁺ helper and CD8⁺ cytotoxic T cells. Such CD4⁺ and CD8⁺ populations can be further sorted into sub-populations by positive or negative selection for markers expressed or expressed to a relatively higher degree on one or more naive, memory, and/or effector T cell subpopulations.

CD8⁺ T cells may be further enriched for or depleted of naive, central memory, effector memory, and/or central memory stem cells, such as by positive or negative selection based on surface antigens associated with the respective subpopulation. Enrichment for central memory T (TC_{M}) cells may be carried out to increase efficacy, such as to improve long-term survival, expansion, and/or engraftment following administration, which in some aspects is particularly robust in such sub-populations.

The T cells may be autologous T cells. In this method, tumor samples are obtained from patients and a single cell suspension is obtained. The single cell suspension can be obtained in any suitable manner, e.g., mechanically (disaggregating the tumor using, e.g., a gentleMACS™ Dissociator, Miltenyi Biotec, Auburn, Calif.) or enzymatically (e.g., collagenase or DNase). Single-cell suspensions of tumor enzymatic digests are cultured in interleukin-2 (IL-2). The cells are cultured until confluence (e.g., about 2×10⁶ lymphocytes), e.g., from about 5 to about 21 days, preferably from about 10 to about 14 days.

The cultured T cells can be pooled and rapidly expanded. Rapid expansion provides an increase in the number of antigen-specific T cells of at least about 50-fold (e.g., 50-, 60-, 70-, 80-, 90-, or 100-fold, or greater) over a period of about 10 to about 14 days. More preferably, rapid expansion provides an increase of at least about 200-fold (e.g., 200-, 300-, 400-, 500-, 600-, 700-, 800-, 900-, or greater) over a period of about 10 to about 14 days.

Expansion can be accomplished by any of a number of methods as are known in the art. For example, T cells can be rapidly expanded using non-specific T-cell receptor stimulation in the presence of feeder lymphocytes and either interleukin-2 (IL-2) or interleukin-15 (IL-15), with IL-2 being preferred. The non-specific T-cell receptor stimulus can include around 30 ng/ml of OKT3, a mouse monoclonal anti-CD3 antibody (available from Ortho-McNeil®, Raritan, N.J.). Alternatively, T cells can be rapidly expanded by stimulation of peripheral blood mononuclear cells (PBMC) in vitro with one or more antigens (including antigenic portions thereof, such as epitope(s), or a cell) of the cancer, which can be optionally expressed from a vector, such as a human leukocyte antigen A2 (HLA-A2) binding peptide, in the presence of a T-cell growth factor, such as 300 IU/mL IL-2 or IL-15, with IL-2 being preferred. The in vitro-induced T cells are rapidly expanded by re-stimulation with the same antigen(s) of the cancer pulsed onto HLA-A2-expressing antigen-presenting cells. Alternatively, the T-cells can be re-stimulated with irradiated, autologous lymphocytes or with irradiated HLA-A2+ allogeneic lymphocytes and IL-2, for example.

The autologous T-cells can be modified to express a T-cell growth factor that promotes the growth and activation of the autologous T-cells. Suitable T-cell growth factors include, for example, interleukin (IL)-2, IL-7, IL-15, and IL-12. Suitable methods of modification are known in the art. See, for instance, Sambrook et al., MOLECULAR CLONING: A LABORATORY MANUAL, 3^{rd} ed., Cold Spring Harbor Press, Cold Spring Harbor, N.Y. 2001; and Ausubel et al., CURRENT PROTOCOLS IN MOLECULAR BIOLOGY, Greene Publishing Associates and John Wiley & Sons, NY, 1994. In particular aspects, modified autologous T-cells express the T-cell growth factor at high levels. T-cell growth factor coding sequences, such as that of IL-12, are readily available in the art, as are promoters, the operable linkage of which to a T-cell growth factor coding sequence promote high-level expression.

### F. Engineering of Immune Effector Cells

The immune effectors cells (e.g., autologous or allogeneic T cells (e.g., regulatory T cells, CD4+ T cells, CD8+ T cells, or gamma-delta T cells)) may be genetically engineered to express antigen receptors such as chimeric antigen receptors (CARs). For example, the host cells (e.g., autologous or allogeneic T-cells) may be modified to express a CAR having antigenic specificity for GPC2. Multiple CARs, such as to different antigens, may be added to a single cell type, such as T cells.

The cells may comprise one or more nucleic acids introduced via genetic engineering that encode one or more antigen receptors, and genetically engineered products of such nucleic acids. The nucleic acids may be heterologous, i.e., normally not present in a cell or sample obtained from the cell, such as one obtained from another organism or cell, which for example, is not ordinarily found in the cell being engineered and/or an organism from which such cell is derived. The nucleic acids may not be naturally occurring, such as a nucleic acid not found in nature (e.g., chimeric).

In some aspects, the engineered immune effector cells are modified to decrease or eliminate the expression of one or more endogenous genes. For example, the engineered immune effector cells may be modified to knock down or knock out at least one immune checkpoint protein. The at least one immune checkpoint gene may be selected from the group consisting of: PD1, CTLA4, LAG3, TIM3, TIGIT, CD96, BTLA, KIRs, adenosine A2a receptor, Vista, IDO, FAS, SIRP alpha, CISH, SHP-1, FOXP3, LAIR1, PVRIG, PPP2CA, PPP2CB, PTPN6, PTPN22, CD160, CRTAM, SIGLEC7, SIGLEC9, CD244, TNFRSF10B, TNFRSF10A, CASP8, CASP10, CASP3, CASP6, CASP7, FADD, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, PRDM1, BATF, GUCY1A2, GUCY1A3, GUCY1B2, and GUCY1B3.

As another example, HLA genes in the engineered immune effector cells may be modified in various ways. For example, the engineered immune effector cells may be engineered such that they do not express functional HLA-A on their surface. The HLA-A negative engineered immune effector cells may be derived from an HLA-homozygous individual. Alternatively, the engineered immune effector cells may be HLA-A homozygous. Further, the engineered immune effector cells, regardless of whether they are HLA-A negative or HLA-A homozygous, may be HLA-homozygous at HLA-B, HLA-C, and/or HLA-DRB1 alleles.

In some aspects, the engineered immune effector cells may be modified to knock down or knock out the expression of one or more T-cell receptor component. For example, in some aspects, the cell lacks expression or have reduced expression of TCRα, TCRβ, TCRα and TCRβ, TCRγ, TCRδ, TCRγ and TCRδ, or any combination of the foregoing. Such can occur by any suitable manner, including by introducing zinc finger nucleases (ZFN), for example, targeting the constant region of one or more of the TCR receptor components.

### G. Methods of Propagating Immune Effector Cells

In some cases, immune effector cells of the embodiments (e.g., T-cells) are co-cultured with activating and propagating cells (AaPCs), to aid in cell expansion. For example, antigen presenting cells (APCs) are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009, each of which is incorporated by reference.

In some cases, AaPCs express an antigen of interest (e.g., GPC2). Furthermore, in some cases, APCs can express an antibody that binds to either a specific CAR polypeptide or to CAR polypeptides in general (e.g., a universal activating and propagating cell (uAPC). Such methods are disclosed in International (PCT) Patent Pub. no. WO/2014/190273, which is incorporated herein by reference. In addition to antigens of interest, the AaPC systems may also comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD70 and B7.1 (B7.1 was previously known as B7 and also known as CD80), which among other things, bind to CD28 and/or CTLA-4 molecules on the surface of T cells, thereby affecting, for example, T-cell expansion, Th1 differentiation, short-term T-cell survival, and cytokine secretion such as interleukin (IL)-2 (see Kim et al., 2004). Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), that promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001, incorporated herein by reference.

Cells selected to become AaPCs, preferably have deficiencies in intracellular antigen-processing, intracellular peptide trafficking, and/or intracellular MHC Class I or Class II molecule-peptide loading, or are poikilothermic (i.e., less sensitive to temperature challenge than mammalian cell lines), or possess both deficiencies and poikilothermic properties. Preferably, cells selected to become AaPCs also lack the ability to express at least one endogenous counterpart (e.g., endogenous MHC Class I or Class II molecule and/or endogenous assisting molecules as described above) to the exogenous MHC Class I or Class II molecule and assisting molecule components that are introduced into the cells. Furthermore, AaPCs preferably retain the deficiencies and poikilothermic properties that were possessed by the cells prior to their modification to generate the AaPCs. Exemplary AaPCs either constitute or are derived from a transporter associated with antigen processing (TAP)-deficient cell line, such as an insect cell line. An exemplary poikilothermic insect cells line is a Drosophila cell line, such as a Schneider 2 cell line (see, e.g., Schneider 1972 Illustrative methods for the preparation, growth, and culture of Schneider 2 cells, are provided in U.S. Pat. Nos. 6,225,042, 6,355,479, and 6,362,001.

In one embodiment, AaPCs are also subjected to a freeze-thaw cycle. In an exemplary freeze-thaw cycle, the AaPCs may be frozen by contacting a suitable receptacle containing the AaPCs with an appropriate amount of liquid nitrogen, solid carbon dioxide (i.e., dry ice), or similar low-temperature material, such that freezing occurs rapidly. The frozen APCs are then thawed, either by removal of the AaPCs from the low-temperature material and exposure to ambient room temperature conditions, or by a facilitated thawing process in which a lukewarm water bath or warm hand is employed to facilitate a shorter thawing time. Additionally, AaPCs may be frozen and stored for an extended period of time prior to thawing. Frozen AaPCs may also be thawed and then lyophilized before further use. Preferably, preservatives that might detrimentally impact the freeze-thaw procedures, such as dimethyl sulfoxide (DMSO), polyethylene glycols (PEGs), and other preservatives, are absent from media containing AaPCs that undergo the freeze-thaw cycle, or are essentially removed, such as by transfer of AaPCs to media that is essentially devoid of such preservatives.

In further embodiments, xenogenic nucleic acid and nucleic acid endogenous to the AaPCs, may be inactivated by crosslinking, so that essentially no cell growth, replication or expression of nucleic acid occurs after the inactivation. In one embodiment, AaPCs are inactivated at a point subsequent to the expression of exogenous MHC and assisting molecules, presentation of such molecules on the surface of the AaPCs, and loading of presented MHC molecules with selected peptide or peptides. Accordingly, such inactivated and selected peptide loaded AaPCs, while rendered essentially incapable of proliferating or replicating, retain selected peptide presentation function. Preferably, the crosslinking also yields AaPCs that are essentially free of contaminating microorganisms, such as bacteria and viruses, without substantially decreasing the antigen-presenting cell function of the AaPCs. Thus crosslinking maintains the important AaPC functions of while helping to alleviate concerns about safety of a cell therapy product developed using the AaPCs. For methods related to crosslinking and AaPCs, see for example, U.S. Patent Application Publication No. 20090017000, which is incorporated herein by reference.

### II. Bispecific Innate Immune Cell Engagers (BICEs)

In one embodiment, provided here is a bispecific innate immune cell engager (BICE) binding both GD2 on tumor cells and CD16A on natural killers (NK) and macrophages. The BICE sequence may be preceded by an Ig Kappa leader motif to enhance secretion (sequence provided in Table 8) and followed by a His-tag element to detect the product in vitro and in vivo. The BICE transgene may be composed of the GD2 antibody 14g2a scFv sequences (Tables 9 and 10) linked to a CD16A single-domain antibody (sdAb; PCT Publn. WO2018/039626, which is incorporated by reference herein in its entirety; Table 11).

TABLE 8
Sequence for Ig Kappa leader

| Region | Sequence | SEQ ID NO: |
|---|---|---|
| Ig Kappa | METDTLLLWVLLLWVPGSTGD | 11 |
| leader | | |

TABLE 9
Sequence for GD2 variable region

| | | SEQ |
|---|---|---|
| | | ID |
| Region | Sequence | NO: |
| VL | DILLTQTPLSLPVSLGDQASISCRSSQSLVHRNGNTYLH | 12 |
| | WYLQKPGQSPKLLIHKVSNRFSGVPDRFSGSGSGTDFTL | |
| | KISRVEAEDLGVYFCSQSTHVPPLTFGAGTKLELK | |
| VH | EVKLQQSGPSLVEPGASVMISCKASGSSFTGYNMNWVRQ | 13 |
| | NIGKSLEWIGAIDPYYGGTSYNQKFKGRATLTVDKSSST | |
| | AYMHLKSLTSEDSAVYYCVSGMEYWGQGTSVTVSS | |

TABLE 10
Exemplary linker sequence used
between VL and VH GD2 scFv

| Region | Sequence | SEQ ID NO: |
|---|---|---|
| Linker | GSTSGSGKPGSGEGSTKG | 14 |

An sdA, which is also known as a domain antibody (dAb) or engineered antibody domain (eAd)), is a fragment consisting of a single monomeric variable antibody domain from the heavy or light chains. In the case of an sdA that targets CD16A, it may be a polypeptide comprising, consisting essentially of, or consisting of the amino acid sequence of SEQ ID NO: 15.

In a particular embodiment, the CD16A single-domain antibody polypeptide comprises, consists essentially of, or consists of the amino acid sequence of SEQ ID NO: 15 (referred to as sdA1), which comprises the complementarity determining region (CDR) sequences (e.g., CDR1, CDR2, and CDR3) of SEQ ID NOs: 16, 17, and 18. The polypeptide may comprise, consist essentially of, or consist of an amino acid sequence having at least 85% (e.g., 86%, 87%, 88%, 89%, 90%, 91%, 92%, 93%, 94%, 95%, 96%, 97%, 98%, or 99%) sequence identity to the amino acid sequence of SEQ ID NO: 15. The variants of sdA1 (SEQ ID NO: 15) may not contain changes to the CDRs described above (i.e., the CDR sequences are maintained without modification in the variants of sdA1).

TABLE 11
Sequences for CD16a sdAb

| | | SEQ |
|---|---|---|
| | | ID |
| Domain | Sequence | NO: |
| sdA1 | EVQLVESGGGLVQPGGSLRLSCAASGFTFSNYGMSWVRQ | 15 |
| | APGKGLEWIGSIYYSGSTNYNPSLKSLVTISRDNSKNTL | |
| | YLQMNSLRAEDTATYYCARESIDYWGQGTLVTVSS | |
| sdA1 | GFTFSNYG | 16 |
| CDR1 | | |
| sdA1 | IYYSGST | 17 |
| CDR2 | | |
| sdA1 | ARESIDY | 18 |
| CDR3 | | |

The GD2 scFv and the anti-CD16A may be joined via a linker (i.e., a flexible molecular connection, such as a flexible polypeptide chain). The linker can be any suitable linker of any length, but is preferably at least about 15 (e.g., at least about 20, at least about 25, at least about 30, at least about 35, at least about 40, at least about 45, at least about 50, or ranges thereof) amino acids in length. In one embodiment, the linker is an amino acid sequence that is naturally present in immunoglobulin molecules of the host, such that the presence of the linker would not result in an immune response against the linker sequence by the mammal. Examples of suitable linkers include, but are not limited to, linkers that comprise one or more (e.g., two, three, four, five, six, seven, eight, nine, or ten) G4S motifs. An exemplary linker sequence is provided in Table 12.

TABLE 12
Exemplary linker sequence used to join GD2
scFv and CD16 a sdAb

| Region | Sequence | SEQ ID NO: |
|---|---|---|
| Linker | GSTSGSGKPGSGEGSTKG | 19 |

A BICE consisting of the GD2 antibody 14g2a scFv sequences linked to a CD16A single-domain antibody may have a sequence as provided in Table 13.

TABLE 13
GD2 scFv and CD16 a sdAb BICE

| | | SEQ |
|---|---|---|
| Region | Sequence | ID NO: |
| BICE | DILLTQTPLSLPVSLGDQASISCRSSQSLVHRNG | 21 |
| | NTYLHWYLQKPGQSPKLLIHKVSNRFSGVPDRFS | |
| | GSGSGTDFTLKISRVEAEDLGVYFCSQSTHVPPL | |
| | TFGAGTKLELKGSTSGSGKPGSGEGSTKGEVKLQ | |
| | QSGPSLVEPGASVMISCKASGSSFTGYNMNWVRQ | |
| | NIGKSLEWIGAIDPYYGGTSYNQKFKGRATLTVD | |
| | KSSSTAYMHLKSLTSEDSAVYYCVSGMEYWGQGT | |
| | SVTVSSGSTSGSGKPGSGEGSTKGEVQLVESGGG | |
| | LVQPGGSLRLSCAASGFTFSNYGMSWVRQAPGKG | |
| | LEWIGSIYYSGSTNYNPSLKSLVTISRDNSKNTL | |
| | YLQMNSLRAEDTATYYCARESIDYWGQGTLVTVS | |
| | S | |

### III. Expression Cassettes

Provided herein are expression cassettes that encode a GPC2 CAR having a CD28-based intracellular stimulatory signaling domain and a GD2-targeted BiCEs. The expression cassette may have a cleavable peptide located between the CAR and the BiCE. In some cases, the cleavable peptide may be a self-cleavable peptide, such as, for example, a 2A peptide. The 2A peptide may be a T2A peptide, a P2A peptide, an E2A peptide, or a F2A peptide. The presence of this peptide provides for separation of the CAR protein from the BiCE following translation. In some cases, the cleavable peptide may be a cleavage site for a widely expressed, endogenous endoprotease, such as, for example, furin, prohormone convertase 7 (PC7), paired basic amino-acid cleaving enzyme 4 (PACE4), or subtilisin kexin isozyme 2 (SKI-1). In some cases, the cleavable peptide may be a cleavage site for a tissue-specific or cell-specific endoprotease. The P2A sequence used between CAR and BiCE is provided in Table 14. The expression cassette may have a sequence as provided in Table 15.

TABLE 14
Sequence of P2A cassette

| Region | Sequence | SEQ ID NO: |
|---|---|---|
| P2A | ATNFSLLKQAGDVEENPGP | 20 |

TABLE 15
Sequence of bicistronic cassette encoding a GPC2
CAR and a GD2-targeted BiCEs

| Sequence | SEQ ID NO: |
|---|---|
| ATGGCCCTGCCAGTGACCGCCCTGCTGCTGCCACTGGCCCTGCTGCTG | 23 |
| CACGCAGCCCGCCCTGACATCCAGATGACACAGAGCCCTTCCACCCT | |
| GAGCGCCTTCGTGGGCGATCGCGTGACAATCACCTGTCGGGCCTCTC | |
| AGAGCATCTCTAGCTGGCTGGCCTGGTACCAGCAGAAGCCAGGCAAG | |
| GCCCCCAAGCTGCTGATCTATGCCGCCTCTACACTGCAGAGCGGCGT | |
| GCCATCCAGGTTCTCCGGATCTGGAAGCGGAACCGAGTTTACACTGA | |
| CCATCTCCTCTCTGCAGCCCGAGGACTTCGCCACATACTATTGCCAGC | |
| AGCTGAATAGCTACCCTATCACATTTGGCCAGGGCACCAGACTGGAG | |
| ATCAAGAGGGGAGGAGGAGGAAGCGGCGGAGGAGGCTCCGGCGGC | |
| GGCGGCTCTGAGGTGCAGCTGGTGGAGACAGGAGGAGGAGTGGTGA | |
| AGCCAGGAGGCAGCCTGAGACTGTCCTGCGCAGCATCTGGCTTCACA | |
| TTTTCCGACTACTATATGTCTTGGATCAGGCAGGCACCAGGCAAGGG | |
| CCTGGAGTGGGTGAGCTACATCAGCTCCTCTGGCTCCACCATCTACTA | |
| TGCCGACTCTGTGAAGGGCCGCTTCACAATCAGCCGGGATAACTCCA | |
| AGAATACCCTGTACCTGCAGATGAACAGCCTGAGAGCCGAGGATACC | |
| GCCGTGTACTATTGCGCCAGGGAGTCCGGCTACGACTACGTGTTCGA | |
| TTATTGGGGCCAGGGCACACTGGTGGCCGTGAGCTCCATTGAAGTTA | |
| TGTATCCTCCTCCTTACCTAGACAATGAGAAGAGCAATGGAACCATT | |
| ATCCATGTGAAAGGGAAACACCTTTGTCCAAGTCCCCTATTTCCCGG | |
| ACCTTCTAAGCCCTTTTGGGTGCTGGTGGTGGTTGGTGGAGTCCTGGC | |
| TTGCTATAGCTTGCTAGTAACAGTGGCCTTTATTATTTTCTGGGTGAG | |
| GAGTAAGAGGAGCAGGCTCCTGCACAGTGACTACATGAACATGACTC | |
| CCCGCCGCCCCGGGCCCACCCGCAAGCATTACCAGCCCTATGCCCCA | |
| CCACGCGACTTCGCAGCCTATAGAGTGAAGTTTTCCAGGTCTGCCGA | |
| TGCACCAGCATACAAGCAGGGACAGAACCAGCTGTATAACGAGCTG | |
| AATCTGGGCCGGAGAGAGGAGTACGACGTGCTGGATAAGAGGCGCG | |
| GCAGAGATCCTGAGATGGGCGGCAAGCCCCGGAGAAAGAACCCTCA | |
| GGAGGGCCTGTACAATGAGCTGCAGAAGGACAAGATGGCCGAGGCC | |
| TATTCTGAGATCGGCATGAAGGGAGAGAGGCGCCGGGGCAAGGGAC | |
| ACGATGGCCTGTACCAGGGCCTGTCCACAGCCACCAAGGACACCTAT | |
| GATGCACTGCACATGCAGGCCCTGCCACCAAGGGCTACTAACTTTTC | |
| TCTTCTCAAACAAGCTGGAGATGTTGAAGAAAATCCTGGACCTATGG | |
| AAACCGATACTCTGCTCCTTTGGGTGCTCCTGCTGTGGGTGCCCGGCA | |
| GCACCGGCGATGACATCTTGCTCACACAAACTCCCTTGTCCCTGCCCG | |
| TCAGTCTTGGAGACCAAGCTTCCATATCTTGTAGAAGTTCTCAAAGTC | |
| TTGTACACCGAAATGGAAATACATACTTGCACTGGTATCTCCAAAAG | |
| CCCGGTCAATCACCCAAGTTGCTCATACACAAGGTCTCTAATCGGTTC | |
| TCTGGAGTCCCCGACCGTTTCAGTGGATCAGGAAGTGGAACAGACTT | |
| CACACTCAAGATATCAAGAGTCGAGGCAGAGGACCTCGGGGTTTACT | |
| TCTGTTCCCAATCAACACACGTCCCACCCTTGACATTCGGAGCCGGA | |
| ACTAAGTTGGAGTTGAAGGGGTCAACATCTGGATCAGGTAAACCAGG | |
| TAGCGGTGAAGGAAGCACTAAAGGTGAGGTCAAGCTTCAACAAAGT | |
| GGTCCCTCTCTTGTCGAGCCTGGGGCTTCCGTTATGATCTCCTGTAAG | |
| GCATCCGGAAGTTCTTTCACTGGATACAATATGAATTGGGTTCGTCAA | |
| AATATCGGGAAGTCTTTGGAGTGGATAGGAGCTATCGACCCTTACTA | |
| CGGCGGTACATCCTACAATCAAAAGTTCAAGGGACGTGCCACTTTGA | |
| CTGTCGACAAGTCTTCCTCTACTGCTTACATGCACCTTAAGTCACTTA | |
| CTTCAGAGGACTCTGCTGTTTACTACTGTGTCAGTGGGATGGAGTACT | |
| GGGGTCAAGGGACTTCCGTAACAGTCTCTTCAGGGTCAACTTCTGGTT | |
| CAGGAAAACCAGGAAGCGGGGAAGGTAGTACTAAAGGAGAGGTCCA | |
| ATTGGTTGAGTCTGGAGGAGGGCTCGTCCAACCAGGTGGAAGTTTGA | |
| GACTTTCATGTGCCGCTTCCGGATTCACATTCTCCAATTACGGTATGT | |
| CTTGGGTACGACAAGCTCCCGGTAAGGGACTCGAGTGGATCGGGTCC | |
| ATATACTACTCTGGGTCCACAAATTACAATCCATCCCTCAAGTCACTT | |
| GTTACAATCTCCCGGGACAATTCAAAGAATACTTTGTACTTGCAAAT | |
| GAATTCTCTTCGGGCAGAGGACACTGCTACTTACTACTGTGCACGAG | |
| AGTCTATAGACTACTGGGGACAAGGGACGTTGGTAACTGTCTCCAGT | |
| CACCATCACCATCACCAT | |

Expression cassettes can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

A “promoter” refers to a nucleotide sequence, usually upstream (5′) of a coding sequence, which directs and/or controls the expression of the coding sequence by providing the recognition for RNA polymerase and other factors required for proper transcription. “Promoter” includes a minimal promoter that is a short DNA sequence comprised of a TATA-box and optionally other sequences that serve to specify the site of transcription initiation, to which regulatory elements are added for control of expression.

An “enhancer” is a DNA sequence that can stimulate transcription activity and may be an innate element of the promoter or a heterologous element that enhances the level or tissue specificity of expression. It is capable of operating in either orientation (5′->3′ or 3′->5′), and may be capable of functioning even when positioned either upstream or downstream of the promoter.

Promoters and/or enhancers may be derived in their entirety from a native gene, or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: human elongation factor-1 alpha (EFla), hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

In some aspects, an expression cassette can be comprised within a viral vector. A viral vector is derived from or based upon one or more nucleic acid elements that comprise a viral genome. Exemplary viral vectors include adeno-associated virus (AAV) vectors, retroviral vectors, and lentiviral vectors.

For example, lentiviruses are complex retroviruses, which, in addition to the common retroviral genes gag, pol, and env, contain other genes with regulatory or structural function. The higher complexity enables the virus to modulate its life cycle, as in the course of latent infection. Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe. Lentiviral vectors are well known in the art (see, e.g., U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell, wherein a suitable host cell is transfected with two or more vectors carrying the packaging functions, namely gag, pol and env, as well as rev and tat, is described in U.S. Pat. No. 5,994,136, incorporated herein by reference.

The lentiviral genome and the proviral DNA have the three genes found in retroviruses: gag, pol and env, which are flanked by two long terminal repeat (LTR) sequences. The gag gene encodes the internal structural (matrix, capsid and nucleocapsid) proteins; the pol gene encodes the RNA-directed DNA polymerase (reverse transcriptase), a protease and an integrase; and the env gene encodes viral envelope glycoproteins. The 5′ and 3′ LTRs serve to promote transcription and polyadenylation of the virion RNAs. The LTR contains all other cis-acting sequences necessary for viral replication. Lentiviruses have additional genes including vif, vpr, tat, rev, vpu, nef and vpx.

Adjacent to the 5′ LTR are sequences necessary for reverse transcription of the genome (the tRNA primer binding site) and for efficient encapsidation of viral RNA into particles (the Psi site). If the sequences necessary for encapsidation (or packaging of retroviral RNA into infectious virions) are missing from the viral genome, the cis defect prevents encapsidation of genomic RNA. However, the resulting mutant remains capable of directing the synthesis of all virion proteins.

### IV. Modifications of Proteins

The sequences of antibodies may be modified for a variety of reasons, such as improved expression, improved cross-reactivity, or diminished off-target binding. Modified antibodies may be made by any technique known to those of skill in the art, including expression through standard molecular biological techniques, or the chemical synthesis of polypeptides.

For example, one may wish to make modifications, such as introducing conservative changes into an antibody molecule. In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

The substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: basic amino acids: arginine (+3.0), lysine (+3.0), and histidine (−0.5); acidic amino acids: aspartate (+3.0±1), glutamate (+3.0±1), asparagine (+0.2), and glutamine (+0.2); hydrophilic, nonionic amino acids: serine (+0.3), asparagine (+0.2), glutamine (+0.2), and threonine (−0.4), sulfur containing amino acids: cysteine (−1.0) and methionine (−1.3); hydrophobic, nonaromatic amino acids: valine (−1.5), leucine (−1.8), isoleucine (−1.8), proline (−0.5±1), alanine (−0.5), and glycine (0); hydrophobic, aromatic amino acids: tryptophan (−3.4), phenylalanine (−2.5), and tyrosine (−2.3).

An amino acid can be substituted for another having a similar hydrophilicity and produce a biologically or immunologically modified protein. In such changes, the substitution of amino acids whose hydrophilicity values are within ±2 is preferred, those that are within ±1 are particularly preferred, and those within ±0.5 are even more particularly preferred.

Amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

The present disclosure also contemplates isotype modification. By modifying the Fc region to have a different isotype, different functionalities can be achieved. For example, changing to IgG1 can increase antibody dependent cell cytotoxicity, switching to class A can improve tissue distribution, and switching to class M can improve valency.

One can design an Fc region of an antibody with altered effector function, e.g., by modifying C1q binding and/or FcγR binding and thereby changing CDC activity and/or ADCC activity. “Effector functions” are responsible for activating or diminishing a biological activity (e.g., in a subject). Examples of effector functions include, but are not limited to: C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved C1q binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

An isolated monoclonal antibody, or antigen binding fragment thereof, may contain a substantially homogeneous glycan without sialic acid, galactose, or fucose. The aforementioned substantially homogeneous glycan may be covalently attached to the heavy chain constant region.

A monoclonal antibody may have a novel Fc glycosylation pattern. Glycosylation of an Fc region is typically either N-linked or O-linked. N-linked refers to the attachment of the carbohydrate moiety to the side chain of an asparagine residue. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation site(s) that are not present in the polypeptide. Addition of glycosylation sites to the Fc region of an antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

The isolated monoclonal antibody, or antigen binding fragment thereof, may be present in a substantially homogenous composition represented by the GNGN or G1/G2 glycoform, which exhibits increased binding affinity for Fc gamma RI and Fc gamma RIII compared to the same antibody without the substantially homogeneous GNGN glycoform and with G0, G1F, G2F, GNF, GNGNF or GNGNFX containing glycoforms. Fc glycosylation plays a significant role in anti-viral and anti-cancer properties of therapeutic mAbs. Elimination of core fucose dramatically improves the ADCC activity of mAbs mediated by natural killer (NK) cells but appears to have the opposite effect on the ADCC activity of polymorphonuclear cells (PMNs).

The isolated monoclonal antibody, or antigen binding fragment thereof, may be expressed in cells that express beta (1,4)-N-acetylglucosaminyltransferase III (GnT III), such that GnT III adds GlcNAc to the antibody. Methods for producing antibodies in such a fashion are provided in WO/9954342 and WO/03011878. Cell lines can be altered to enhance or reduce or eliminate certain post-translational modifications, such as glycosylation, using genome editing technology such as Clustered Regularly Interspaced Short Palindromic Repeats (CRISPR). For example, CRISPR technology can be used to eliminate genes encoding glycosylating enzymes in 293 or CHO cells used to express monoclonal antibodies.

It is possible to engineer the antibody variable gene sequences obtained from human B cells to enhance their manufacturability and safety. Potential protein sequence liabilities can be identified by searching for sequence motifs associated with sites containing:
1) Unpaired Cys residues,
2) N-linked glycosylation,
3) Asn deamidation,
4) Asp isomerization,
5) SYE truncation,
6) Met oxidation,
7) Trp oxidation,
8) N-terminal glutamate,
9) Integrin binding,
10) CD11c/CD18 binding, or
11) Fragmentation
   Such motifs can be eliminated by altering the synthetic gene comprising the cDNA encoding the antibodies.

Antibodies can be engineered to enhance solubility. For example, some hydrophilic residues such as aspartic acid, glutamic acid, and serine contribute significantly more favorably to protein solubility than other hydrophilic residues, such as asparagine, glutamine, threonine, lysine, and arginine.

B cell repertoire deep sequencing of human B cells from blood donors has been performed on a wide scale. Sequence information about a significant portion of the human antibody repertoire facilitates statistical assessment of antibody sequence features common in healthy humans. With knowledge about the antibody sequence features in a human recombined antibody variable gene reference database, the position specific degree of “Human Likeness” (HL) of an antibody sequence can be estimated. HL has been shown to be useful for the development of antibodies in clinical use, like therapeutic antibodies or antibodies as vaccines. The goal is to increase the human likeness of antibodies to reduce potential adverse effects and anti-antibody immune responses that will lead to significantly decreased efficacy of the antibody drug or can induce serious health implications. One can assess antibody characteristics of the combined antibody repertoire of three healthy human blood donors of about 400 million sequences in total and created a novel “relative Human Likeness” (rHL) score that focuses on the hypervariable region of the antibody. The rHL score allows one to easily distinguish between human (positive score) and non-human sequences (negative score). Antibodies can be engineered to eliminate residues that are not common in human repertoires.

Methods for reducing or eliminating the antigenicity of antibodies and antibody fragments are known in the art. When the antibodies are to be administered to a human, the antibodies preferably are “humanized” to reduce or eliminate antigenicity in humans. Preferably, each humanized antibody has the same or substantially the same affinity for the antigen as the non-humanized mouse antibody from which it was derived.

In one humanization approach, chimeric proteins are created in which mouse immunoglobulin constant regions are replaced with human immunoglobulin constant regions. See, e.g., Morrison et al., 1984, PROC. NAT. ACAD. SCI. 81:6851-6855, Neuberger et al., 1984, NATURE 312:604-608; U.S. Pat. No. 6,893,625 (Robinson); U.S. Pat. No. 5,500,362 (Robinson); and U.S. Pat. No. 4,816,567 (Cabilly).

In an approach known as CDR grafting, the CDRs of the light and heavy chain variable regions are grafted into frameworks from another species. For example, murine CDRs can be grafted into human FRs. In some embodiments, the CDRs of the light and heavy chain variable regions of an antibody are grafted into human FRs or consensus human FRs. To create consensus human FRs, FRs from several human heavy chain or light chain amino acid sequences are aligned to identify a consensus amino acid sequence. CDR grafting is described in U.S. Pat. No. 7,022,500 (Queen); U.S. Pat. No. 6,982,321 (Winter); U.S. Pat. No. 6,180,370 (Queen); U.S. Pat. No. 6,054,297 (Carter); U.S. Pat. No. 5,693,762 (Queen); U.S. Pat. No. 5,859,205 (Adair); U.S. Pat. No. 5,693,761 (Queen); U.S. Pat. No. 5,565,332 (Hoogenboom); U.S. Pat. No. 5,585,089 (Queen); U.S. Pat. No. 5,530,101 (Queen); Jones et al. (1986) NATURE 321:522-525; Riechmann et al. (1988) NATURE 332:323-327; Verhoeyen et al. (1988) SCIENCE 239:1534-1536; and Winter (1998) FEBS LETT 430:92-94.

In an approach called “SUPERHUMANIZATION™,” human CDR sequences are chosen from human germline genes, based on the structural similarity of the human CDRs to those of the mouse antibody to be humanized. See, e.g., U.S. Pat. No. 6,881,557 (Foote); and Tan et al., 2002, J. IMMUNOL. 169:1119-1125.

Other methods to reduce immunogenicity include “reshaping,” “hyperchimerization,” and “veneering/resurfacing.” See, e.g., Vaswami et al., 1998, ANNALS OF ALLERGY, ASTHMA, & IMMUNOL. 81:105; Roguska et al., 1996, PROT. ENGINEER 9:895-904; and U.S. Pat. No. 6,072,035 (Hardman). In the veneering/resurfacing approach, the surface accessible amino acid residues in the murine antibody are replaced by amino acid residues more frequently found at the same positions in a human antibody. This type of antibody resurfacing is described, e.g., in U.S. Pat. No. 5,639,641 (Pedersen).

Another approach for converting a mouse antibody into a form suitable for medical use in humans is known as ACTIVMAB™ technology (Vaccinex, Inc., Rochester, NY), which involves a vaccinia virus-based vector to express antibodies in mammalian cells. High levels of combinatorial diversity of IgG heavy and light chains can be produced. See, e.g., U.S. Pat. No. 6,706,477 (Zauderer); U.S. Pat. No. 6,800,442 (Zauderer); and U.S. Pat. No. 6,872,518 (Zauderer). Another approach for converting a mouse antibody into a form suitable for use in humans is technology practiced commercially by KaloBios Pharmaceuticals, Inc. (Palo Alto, CA). This technology involves the use of a proprietary human “acceptor” library to produce an “epitope focused” library for antibody selection. Another approach for modifying a mouse antibody into a form suitable for medical use in humans is HUMAN ENGINEERING™ technology, which is practiced commercially by XOMA (US) LLC. See, e.g., International (PCT) Publication No. WO 93/11794 and U.S. Pat. No. 5,766,886 (Studnicka); U.S. Pat. No. 5,770,196 (Studnicka); U.S. Pat. No. 5,821,123 (Studnicka); and U.S. Pat. No. 5,869,619 (Studnicka).

Any suitable approach, including any of the above approaches, can be used to reduce or eliminate human immunogenicity of an antibody.

### V. Methods of Treatment

In some aspects, the constructs and cells of the embodiments find application in subjects having or suspected of having a cancer. Suitable immune effector cells that can be used include cytotoxic lymphocytes (CTL). As is well-known to one of skill in the art, various methods are readily available for isolating these cells from a subject. For example, using cell surface marker expression or using commercially available kits (e.g., ISOCELL™ from Pierce, Rockford, Ill.).

Once it is established that the transfected or transduced immune effector cell (e.g., T cell) is capable of expressing the chimeric antigen receptor as a surface membrane protein and secreting the BiCE with the desired regulation and at a desired level, it can be determined whether the chimeric antigen receptor is functional in the host cell to provide for the desired signal induction. Subsequently, the transduced immune effector cells are reintroduced or administered to the subject to activate anti-tumor responses in the subject. To facilitate administration, the transduced T cells according to the embodiments can be made into a pharmaceutical composition or made into an implant appropriate for administration in vivo, with appropriate carriers or diluents, which further can be pharmaceutically acceptable. The means of making such a composition or an implant have been described in the art (see, for instance, Remington's Pharmaceutical Sciences, 16th Ed., Mack, ed. (1980)). Where appropriate, the transduced T cells can be formulated into a preparation in semisolid or liquid form, such as a capsule, solution, injection, inhalant, or aerosol, in the usual ways for their respective route of administration. Means known in the art can be utilized to prevent or minimize release and absorption of the composition until it reaches the target tissue or organ, or to ensure timed-release of the composition. Desirably, however, a pharmaceutically acceptable form is employed that does not in effectuate the cells expressing the chimeric antigen receptor. Thus, desirably the transduced T cells can be made into a pharmaceutical composition containing a balanced salt solution, preferably Hanks' balanced salt solution, or normal saline.

In certain embodiments, CAR-expressing cells of the embodiments are delivered to an individual in need thereof, such as an individual that has cancer or an infection. The cells then enhance the individual's immune system to attack the respective cancer. In some cases, the individual is provided with one or more doses of the antigen-specific CAR cells. In cases where the individual is provided with two or more doses of the antigen-specific CAR cells, the duration between the administrations should be sufficient to allow time for propagation in the individual, and in specific embodiments the duration between doses is 1, 2, 3, 4, 5, 6, 7, or more days. Suitable doses for a therapeutic effect would be at least 10⁵ or between about 10⁵ and about 10¹⁰ cells per dose, for example, preferably in a series of dosing cycles. An exemplary dosing regimen consists of four one-week dosing cycles of escalating doses, starting at least at about 10⁵ cells on Day 0, for example increasing incrementally up to a target dose of about 10¹⁰ cells within several weeks of initiating an intra-patient dose escalation scheme. Suitable modes of administration include intravenous, subcutaneous, intracavitary (for example by reservoir-access device), intraperitoneal, and direct injection into a tumor mass.

A pharmaceutical composition of the embodiments (e.g., comprising CAR-expressing T-cells) can be used alone or in combination with other well-established agents useful for treating cancer. Whether delivered alone or in combination with other agents, the pharmaceutical composition of the embodiments can be delivered via various routes and to various sites in a mammalian, particularly human, body to achieve a particular effect. One skilled in the art will recognize that, although more than one route can be used for administration, a particular route can provide a more immediate and more effective reaction than another route. Local or systemic delivery can be accomplished by administration comprising application or instillation of the formulation into body cavities, inhalation or insufflation of an aerosol, or by parenteral introduction, comprising intramuscular, intravenous, intraportal, intrahepatic, peritoneal, subcutaneous, or intradermal administration.

A composition of the embodiments can be provided in unit dosage form wherein each dosage unit, e.g., an injection, contains a predetermined amount of the composition, alone or in appropriate combination with other active agents. The term unit dosage form as used herein refers to physically discrete units suitable as unitary dosages for human and animal subjects, each unit containing a predetermined quantity of the composition of the embodiments, alone or in combination with other active agents, calculated in an amount sufficient to produce the desired effect, in association with a pharmaceutically acceptable diluent, carrier, or vehicle, where appropriate. The specifications for the novel unit dosage forms of the embodiments depend on the particular pharmacodynamics associated with the pharmaceutical composition in the particular subject.

Desirably an effective amount or sufficient number of the isolated transduced T cells is present in the composition and introduced into the subject such that long-term, specific, anti-tumor responses are established to reduce the size of a tumor or eliminate tumor growth or regrowth than would otherwise result in the absence of such treatment. Desirably, the amount of transduced T cells reintroduced into the subject causes a 10%, 20%, 30%, 40%, 50%, 60%, 70%, 80%, 90%, 95%, 98%, or 100% decrease in tumor size when compared to otherwise same conditions wherein the transduced T cells are not present. As used herein the term “anti-tumor effective amount” refers to an effective amount of CAR-expressing immune effector cells to reduce cancer cell or tumor growth in a subject.

Accordingly, the amount of transduced immune effector cells (e.g., T cells) administered should take into account the route of administration and should be such that a sufficient number of the transduced immune effector cells will be introduced so as to achieve the desired therapeutic response. Furthermore, the amounts of each active agent included in the compositions described herein (e.g., the amount per each cell to be contacted or the amount per certain body weight) can vary in different applications. In general, the concentration of transduced T cells desirably should be sufficient to provide in the subject being treated at least from about 1×10⁶ to about 1×10⁹ transduced T cells, even more desirably, from about 1×10⁷ to about 5×10⁸ transduced T cells, although any suitable amount can be utilized either above, e.g., greater than 5×10⁸ cells, or below, e.g., less than 1×10⁷ cells. The dosing schedule can be based on well-established cell-based therapies (see, e.g., U.S. Pat. No. 4,690,915), or an alternate continuous infusion strategy can be employed.

These values provide general guidance of the range of transduced T cells to be utilized by the practitioner upon optimizing the method of the embodiments. The recitation herein of such ranges by no means precludes the use of a higher or lower amount of a component, as might be warranted in a particular application. For example, the actual dose and schedule can vary depending on whether the compositions are administered in combination with other pharmaceutical compositions, or depending on interindividual differences in pharmacokinetics, drug disposition, and metabolism. One skilled in the art readily can make any necessary adjustments in accordance with the exigencies of the particular situation.

Certain aspects of the present embodiments can be used to prevent or treat a cancer, such as lung cancer, prostate cancer, stomach cancer, thyroid cancer, breast cancer multiple myeloma, melanoma, colon cancer, or pancreatic cancer. “Treatment” and “treating” refer to administration or application of a therapeutic agent to a subject or performance of a procedure or modality on a subject for the purpose of obtaining a therapeutic benefit of a disease or health-related condition. For example, a treatment may include administration of a pharmaceutically effective amount of an anti-GPC2 CAR T cell that secrets a GD2-CD16A BiCE, either alone or in combination with administration of chemotherapy, immunotherapy, or radiotherapy, performance of surgery, or any combination thereof.

The term “subject” as used herein refers to any individual or patient to which the subject methods are performed. Generally, the subject is human, although as will be appreciated by those in the art, the subject may be an animal. Thus, other animals, including mammals, such as rodents (including mice, rats, hamsters, and guinea pigs), cats, dogs, rabbits, farm animals (including cows, horses, goats, sheep, pigs, etc.), and primates (including monkeys, chimpanzees, orangutans, and gorillas) are included within the definition of subject.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, a reduction in the size of a tumor, a reduction in the invasiveness of a tumor, reduction in the growth rate of the cancer, or prevention of metastasis. Treatment of cancer may also refer to prolonging survival of a subject with cancer.

The term “cancer,” as used herein, may be used to describe a solid tumor, metastatic cancer, or non-metastatic cancer. In certain embodiments, the cancer may originate in the bladder, blood, bone, bone marrow, brain, breast, colon, esophagus, duodenum, small intestine, large intestine, colon, rectum, anus, gum, head, kidney, liver, lung, nasopharynx, neck, ovary, pancreas, prostate, skin, stomach, testis, tongue, or uterus.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In certain embodiments, the compositions and methods of the present embodiments involve an adoptive T cell therapy, in combination with a second or additional therapy, such as chemotherapy or immunotherapy.

An adoptive T cell therapy may be administered before, during, after, or in various combinations relative to an anti-cancer treatment. The administrations may be in intervals ranging from concurrently to minutes to days to weeks. In embodiments where the adoptive T cell therapy is provided to a patient separately from an anti-cancer agent, one would generally ensure that a significant period of time did not expire between the time of each delivery, such that the two compounds would still be able to exert an advantageously combined effect on the patient. In such instances, it is contemplated that one may provide a patient with the adoptive T cell therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In certain embodiments, a course of treatment will last 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there is a period of time at which no anti-cancer treatment is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

Various combinations may be employed. For the example below an adoptive T cell therapy is “A” and an anti-cancer therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

### A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DFMO); retinoids, such as retinoic acid; capecitabine; carboplatin, procarbazine, plicomycin, gemcitabien, navelbine, farnesyl-protein transferase inhibitors, transplatinum, and pharmaceutically acceptable salts, acids, or derivatives of any of the above.

### B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2,000 to 6,000 roentgens. Dosage ranges for radioisotopes vary widely, and depend on the half-life of the isotope, the strength and type of radiation emitted, and the uptake by the neoplastic cells.

### C. Immunotherapy

The skilled artisan will understand that immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include B-cell maturation antigen, CD20, carcinoembryonic antigen, tyrosinase (p97), gp68, GPRC5D, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some aspects, a combination described herein includes an agent that decreases tumor immunosuppression, such as a chemokine (C-X-C motif) receptor 2 (CXCR2) inhibitor. In some embodiments, the CXCR2 inhibitor is danirixin (CAS Registry Number: 954126-98-8). Danirixin is also known as GSK1325756 or 1-(4-chloro-2-hydroxy-3-piperidin-3-ylsulfonylphenyl)-3-(3-fluoro-2-methylphenyl) urea. Danirixin is disclosed, e.g., in Miller et al. Eur J Drug Metab Pharmacokinet (2014) 39:173-181; and Miller et al. BMC Pharmacology and Toxicology (2015), 16:18. In some embodiments, the CXCR2 inhibitor is reparixin (CAS Registry Number: 266359-83-5). Reparixin is also known as repertaxin or (2R)-2-[4-(2-methylpropyl)phenyl]-N-methylsulfonylpropanamide. Reparixin is a non-competitive allosteric inhibitor of CXCR1/2. Reparixin is disclosed, e.g., in Zarbock et al. British Journal of Pharmacology (2008), 1-8. In some embodiments, the CXCR2 inhibitor is navarixin. Navarixin is also known as MK-7123, SCH527123, PS291822, or 2-hydroxy-N,N-dimethyl-3-[[2-[[(1R)-1-(5-methylfuran-2-yl) propyl]amino]-3,4-dioxocyclobuten-1-yl]amino]benzamide Navarixin is disclosed, e.g., in Ning et al. Mol Cancer Ther. 2012; 11 (6): 1353-64. In some embodiments, the CXCR2 inhibitor is AZD5069, also known as N-[2-[[(2,3-difluoropheny)methyl]thio]-6-{[(1R,2S)-2,3-dihydroxy-1-methylpropyl]oxy}-4-pyrimidinyl]-1-azetidinesulfonamide. In some embodiments, the CXCR2 inhibitor is an anti-CXCR2 antibody, such as those disclosed in WO2020/028479.

In some aspects, a combination described herein includes an agent that activates dendritic cells, such as, for example, a TLR agonist. A “TLR agonist” as defined herein is any molecule which activates a toll-like receptor as described in Bauer et al., 2001, Proc. Natl. Acad. Sci. USA 98:9237-9242. A TLR agonist may be a small molecule, a recombinant protein, an antibody or antibody fragment, a nucleic acid, or a protein. In certain embodiments, the TLR agonist is recombinant, a natural ligand, an immunostimulatory nucleotide sequence, a small molecule, a purified bacterial extract or an inactivated bacteria preparation.

Several agonists of TLR derived from microbes have been described, such as lipopolysaccharides, peptidoglycans, flagellin and lipoteichoic acid (Aderem et al., 2000, Nature 406:782-787; Akira et al., 2001, Nat. Immunol. 2:675-680) Some of these ligands can activate different dendritic cell subsets, that express distinct patterns of TLRs (Kadowaki et al., 2001, J. Exp. Med. 194:863-869). Therefore, a TLR agonist could be any preparation of a microbial agent that possesses TLR agonist properties. Certain types of untranslated DNA have been shown to stimulate immune responses by activating TLRs. In particular, immunostimulatory oligonucleotides containing CpG motifs have been widely disclosed and reported to activate lymphocytes (see, U.S. Pat. No. 6,194,388). A “CpG motif” as used herein is defined as an unmethylated cytosine-guanine (CpG) dinucleotide. Immunostimulatory oligonucleotides which contain CpG motifs can also be used as TLR agonists according to the methods of the present invention. The immunostimulatory nucleotide sequence may be stabilized by structure modification such as phosphorothioate modification or may be encapsulated in cationic liposomes to improve in vivo pharmacokinetics and tumor targeting.

In some embodiments, the immunotherapy may be an immune checkpoint inhibitor. Immune checkpoints either turn up a signal (e.g., co-stimulatory molecules) or turn down a signal. Immune checkpoints either turn up a signal (e.g., co-stimulatory molecules) or turn down a signal. Immune checkpoint proteins that may be targeted by immune checkpoint blockade include adenosine A2A receptor (A2AR), B7-H3 (also known as CD276), B and T lymphocyte attenuator (BTLA), CCL5, CD27, CD38, CD8A, CMKLR1, cytotoxic T-lymphocyte-associated protein 4 (CTLA-4, also known as CD152), CXCL9, CXCR5, glucocorticoid-induced tumor necrosis factor receptor-related protein (GITR), HLA-DRB1, ICOS (also known as CD278), HLA-DQA1, HLA-E, indoleamine 2,3-dioxygenase 1 (IDO1), killer-cell immunoglobulin (KIR), lymphocyte activation gene-3 (LAG-3, also known as CD223), Mer tyrosine kinase (MerTK), NKG7, OX40 (also known as CD134), programmed death 1 (PD-1), programmed death-ligand 1 (PD-L1, also known as CD274), PDCDILG2, PSMB10, STAT1, T cell immunoreceptor with Ig and ITIM domains (TIGIT), T-cell immunoglobulin domain and mucin domain 3 (TIM-3), and V-domain Ig suppressor of T cell activation (VISTA, also known as C10orf54). In particular, the immune checkpoint inhibitors target the PD-1 axis and/or CTLA-4.

The immune checkpoint inhibitors may be drugs, such as small molecules, recombinant forms of ligand or receptors, or antibodies, such as human antibodies (e.g., International Patent Publication WO2015/016718; Pardoll, Nat Rev Cancer, 12 (4): 252-264, 2012; both incorporated herein by reference). Known inhibitors of the immune checkpoint proteins or analogs thereof may be used, in particular chimerized, humanized, or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

In some embodiments, a PD-1 binding antagonist is a molecule that inhibits the binding of PD-1 to its ligand binding partners. In a specific aspect, the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, a PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or an oligopeptide. Exemplary antibodies are described in U.S. Pat. Nos. 8,735,553, 8,354,509, and 8,008,449, all of which are incorporated herein by reference. Other PD-1 axis antagonists for use in the methods provided herein are known in the art, such as described in U.S. Patent Application Publication Nos. 2014/0294898, 2014/022021, and 2011/0008369, all of which are incorporated herein by reference.

In some embodiments, a PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PD-L2-Fc fusion soluble receptor described in WO2010/027827 and WO2011/066342.

Another immune checkpoint protein that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA-4 is similar to the T-cell co-stimulatory protein, CD28, and both molecules bind to CD80 and CD86, also called B7-1 and B7-2 respectively, on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide. Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in U.S. Pat. No. 8,119,129; PCT Publn. Nos. WO 01/14424, WO 98/42752, WO 00/37504 (CP675,206, also known as tremelimumab; formerly ticilimumab); U.S. Pat. No. 6,207,156; Hurwitz et al. (1998) Proc Natl Acad Sci USA, 95 (17): 10067-10071; Camacho et al. (2004) J Clin Oncology, 22 (145): Abstract No. 2505 (antibody CP-675206); and Mokyr et al. (1998) Cancer Res, 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2, and CDR3 domains of the VL region of ipilimumab. In another embodiment, the antibody competes for binding with and/or binds to the same epitope on CTLA-4 as the above-mentioned antibodies. In another embodiment, the antibody has an at least about 90% variable region amino acid sequence identity with the above-mentioned antibodies (e.g., at least about 90%, 95%, or 99% variable region identity with ipilimumab). Other molecules for modulating CTLA-4 include CTLA-4 ligands and receptors such as described in U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

Another immune checkpoint protein that can be targeted in the methods provided herein is lymphocyte-activation gene 3 (LAG-3), also known as CD223. The complete protein sequence of human LAG-3 has the Genbank accession number NP-002277. LAG-3 is found on the surface of activated T cells, natural killer cells, B cells, and plasmacytoid dendritic cells. LAG-3 acts as an “off” switch when bound to MHC class II on the surface of antigen-presenting cells. Inhibition of LAG-3 both activates effector T cells and inhibitor regulatory T cells. In some embodiments, the immune checkpoint inhibitor is an anti-LAG-3 antibody (e.g., a human antibody, a humanized antibody, or a chimeric antibody), an antigen binding fragment thereof, an immunoadhesin, a fusion protein, or oligopeptide. Anti-human-LAG-3 antibodies (or VH and/or VL domains derived therefrom) suitable for use in the present methods can be generated using methods well known in the art. Alternatively, art recognized anti-LAG-3 antibodies can be used. An exemplary anti-LAG-3 antibody is relatlimab (also known as BMS-986016) or antigen binding fragments and variants thereof (see, e.g., WO 2015/116539). Other exemplary anti-LAG-3 antibodies include TSR-033 (see, e.g., WO 2018/201096), MK-4280, and REGN3767. MGD013 is an anti-LAG-3/PD-1 bispecific antibody described in WO 2017/019846. FS118 is an anti-LAG-3/PD-L1 bispecific antibody described in WO 2017/220569.

Another immune checkpoint protein that can be targeted in the methods provided herein is V-domain Ig suppressor of T cell activation (VISTA), also known as C10orf54. The complete protein sequence of human VISTA has the Genbank accession number NP_071436. VISTA is found on white blood cells and inhibits T cell effector function. In some embodiments, the immune checkpoint inhibitor is an anti-VISTA3 antibody (e.g., a human antibody, a humanized antibody, or a chimeric antibody), an antigen binding fragment thereof, an immunoadhesin, a fusion protein, or oligopeptide. Anti-human-VISTA antibodies (or VH and/or VL domains derived therefrom) suitable for use in the present methods can be generated using methods well known in the art. Alternatively, art recognized anti-VISTA antibodies can be used. An exemplary anti-VISTA antibody is JNJ-61610588 (also known as onvatilimab) (see, e.g., WO 2015/097536, WO 2016/207717, WO 2017/137830, WO 2017/175058). VISTA can also be inhibited with the small molecule CA-170, which selectively targets both PD-L1 and VISTA (see, e.g., WO 2015/033299, WO 2015/033301).

Another immune checkpoint protein that can be targeted in the methods provided herein is indoleamine 2,3-dioxygenase (IDO). The complete protein sequence of human IDO has Genbank accession number NP_002155. In some embodiments, the immune checkpoint inhibitor is a small molecule IDO inhibitor. Exemplary small molecules include BMS-986205, epacadostat (INCB24360), and navoximod (GDC-0919).

Another immune checkpoint protein that can be targeted in the methods provided herein is CD38. The complete protein sequence of human CD38 has Genbank accession number NP_001766. In some embodiments, the immune checkpoint inhibitor is an anti-CD38 antibody (e.g., a human antibody, a humanized antibody, or a chimeric antibody), an antigen binding fragment thereof, an immunoadhesin, a fusion protein, or oligopeptide. Anti-human-CD38 antibodies (or VH and/or VL domains derived therefrom) suitable for use in the present methods can be generated using methods well known in the art. Alternatively, art recognized anti-CD38 antibodies can be used. An exemplary anti-CD38 antibody is daratumumab (see, e.g., U.S. Pat. No. 7,829,673).

Another immune checkpoint protein that can be targeted in the methods provided herein is ICOS, also known as CD278. The complete protein sequence of human ICOS has Genbank accession number NP_036224. In some embodiments, the immune checkpoint inhibitor is an anti-ICOS antibody (e.g., a human antibody, a humanized antibody, or a chimeric antibody), an antigen binding fragment thereof, an immunoadhesin, a fusion protein, or oligopeptide. Anti-human-ICOS antibodies (or VH and/or VL domains derived therefrom) suitable for use in the present methods can be generated using methods well known in the art. Alternatively, art recognized anti-ICOS antibodies can be used. Exemplary anti-ICOS antibodies include JTX-2011 (see, e.g., WO 2016/154177, WO 2018/187191) and GSK3359609 (see, e.g., WO 2016/059602).

Another immune checkpoint protein that can be targeted in the methods provided herein is T cell immunoreceptor with Ig and ITIM domains (TIGIT). The complete protein sequence of human TIGIT has Genbank accession number NP_776160. In some embodiments, the immune checkpoint inhibitor is an anti-TIGIT antibody (e.g., a human antibody, a humanized antibody, or a chimeric antibody), an antigen binding fragment thereof, an immunoadhesin, a fusion protein, or oligopeptide. Anti-human-TIGIT antibodies (or VH and/or VL domains derived therefrom) suitable for use in the present methods can be generated using methods well known in the art. Alternatively, art recognized anti-TIGIT antibodies can be used. An exemplary anti-TIGIT antibody is MK-7684 (see, e.g., WO 2017/030823, WO 2016/028656).

Another immune checkpoint protein that can be targeted in the methods provided herein is OX40, also known as CD134. The complete protein sequence of human OX40 has Genbank accession number NP_003318. In some embodiments, the immune checkpoint inhibitor is an anti-OX40 antibody (e.g., a human antibody, a humanized antibody, or a chimeric antibody), an antigen binding fragment thereof, an immunoadhesin, a fusion protein, or oligopeptide. Anti-human-OX40 antibodies (or VH and/or VL domains derived therefrom) suitable for use in the present methods can be generated using methods well known in the art. Alternatively, art recognized anti-OX40 antibodies can be used. An exemplary anti-OX40 antibody is PF-04518600 (see, e.g., WO 2017/130076). ATOR-1015 is a bispecific antibody targeting CTLA4 and OX40 (see, e.g., WO 2017/182672, WO 2018/091740, WO 2018/202649, WO 2018/002339).

Another immune checkpoint protein that can be targeted in the methods provided herein is glucocorticoid-induced tumor necrosis factor receptor-related protein (GITR), also known as TNFRSF18 and AITR. The complete protein sequence of human GITR has Genbank accession number NP_004186. In some embodiments, the immune checkpoint inhibitor is an anti-GITR antibody (e.g., a human antibody, a humanized antibody, or a chimeric antibody), an antigen binding fragment thereof, an immunoadhesin, a fusion protein, or oligopeptide. Anti-human-GITR antibodies (or VH and/or VL domains derived therefrom) suitable for use in the present methods can be generated using methods well known in the art. Alternatively, art recognized anti-GITR antibodies can be used. An exemplary anti-GITR antibody is TRX518 (see, e.g., WO 2006/105021).

In some embodiment, the immune therapy could be adoptive immunotherapy, which involves the transfer of autologous antigen-specific T cells generated ex vivo. The T cells used for adoptive immunotherapy can be generated either by expansion of antigen-specific T cells or redirection of T cells through genetic engineering (Park, Rosenberg et al. 2011). Isolation and transfer of tumor specific T cells has been shown to be successful in treating melanoma. Novel specificities in T cells have been successfully generated through the genetic transfer of transgenic T cell receptors or chimeric antigen receptors (CARs) (Jena, Dotti et al. 2010). CARs are synthetic receptors consisting of a targeting moiety that is associated with one or more signaling domains in a single fusion molecule. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signaling domains for first generation CARs are derived from the cytoplasmic region of the CD3zeta or the Fc receptor gamma chains. CARs have successfully allowed T cells to be redirected against antigens expressed at the surface of tumor cells from various malignancies including lymphomas and solid tumors (Jena, Dotti et al. 2010).

In one embodiment, the present application provides for a combination therapy for the treatment of cancer wherein the combination therapy comprises adoptive T cell therapy and a checkpoint inhibitor. In one aspect, the adoptive T cell therapy comprises autologous and/or allogenic T-cells. In another aspect, the autologous and/or allogenic T-cells are targeted against tumor antigens.

### D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VI. Definitions

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also fragments thereof (such as Fab, Fab′, F(ab′)₂, Fv, Fd, Fd′, single chain antibody (ScFv), diabody, linear antibody), mutants thereof, naturally occurring variants, fusion proteins comprising an antibody portion with an antigen recognition site of the required specificity, humanized antibodies, chimeric antibodies, and any other modified configuration of the immunoglobulin molecule that comprises an antigen recognition site of the required specificity.

An “isolated antibody” is an antibody that has been separated and/or recovered from a component of its natural environment. Contaminant components of its natural environment are materials that would interfere with diagnostic or therapeutic uses for the antibody, and may include enzymes, hormones, and other proteinaceous or non-proteinaceous solutes. In particular instances, the antibody is purified: (1) to greater than 95% by weight of antibody as determined by the Lowry method, and most particularly more than 99% by weight; or (2) to homogeneity by SDS-PAGE under reducing or non-reducing conditions using Coomassie blue or silver stain. Isolated antibody includes the antibody in situ within recombinant cells since at least one component of the antibody's natural environment will not be present. Ordinarily, however, an isolated antibody will be prepared by at least one purification step.

The basic four-chain antibody unit is a heterotetrameric glycoprotein composed of two identical light (L) chains and two identical heavy (H) chains. The term “heavy chain” as used herein refers to the larger immunoglobulin subunit which associates, through its amino terminal region, with the immunoglobulin light chain. The heavy chain comprises a variable region (V_{H}) and a constant region (CH). The constant region further comprises the C_{H}1, hinge, C_{H}2, and C_{H}3 domains. In the case of IgE, IgM, and IgY, the heavy chain comprises a C_{H}4 domain but does not have a hinge domain. Those skilled in the art will appreciate that heavy chains are classified as gamma, mu, alpha, delta, or epsilon (γ, μ, α, δ, ε), with some subclasses among them (e.g., γ1-γ4, α1-α2). It is the nature of this chain that determines the “class” of the antibody as IgG, IgM, IgA IgD, or IgE, respectively. The immunoglobulin subclasses (isotypes), e.g., IgG1, IgG2, IgG3, IgG4, IgA1, etc. are well characterized and are known to confer functional specialization.

The term “light chain” as used herein refers to the smaller immunoglobulin subunit which associates with the amino terminal region of a heavy chain. As with a heavy chain, a light chain comprises a variable region (V_{L}) and a constant region (C_{L}). Light chains are classified as either kappa or lambda (κ, λ) based on the amino acid sequences of their constant domains (C_{L}). A pair of these can associate with a pair of any of the various heavy chains to form an immunoglobulin molecule. Also encompassed in the meaning of light chain are light chains with a lambda variable region (V-lambda) linked to a kappa constant region (C-kappa) or a kappa variable region (V-kappa) linked to a lambda constant region (C-lambda).

An IgM antibody, for example, consists of 5 basic heterotetramer units along with an additional polypeptide called J chain, and therefore contains 10 antigen binding sites, while secreted IgA antibodies can polymerize to form polyvalent assemblages comprising 2-5 of the basic 4-chain units along with J chain. In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable region (V_{H}) followed by three constant domains (C_{H}) for each of the alpha and gamma chains and four CH domains for mu and isotypes. Each L chain has at the N-terminus, a variable region (V_{L}) followed by a constant domain (C_{L}) at its other end. The V_{L} is aligned with the V_{H} and the C_{L} is aligned with the first constant domain of the heavy chain (C_{H}1). Particular amino acid residues are believed to form an interface between the light chain and heavy chain variable regions. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71, and Chapter 6.

A “variable region” of an antibody refers to the variable region of the antibody light chain or the variable region of the antibody heavy chain, either alone or in combination. The term “variable” refers to the fact that certain segments of the variable regions differ extensively in sequence among antibodies. The variable regions of both the light (VL) and heavy (V_{H}) chain portions mediate antigen binding and define the specificity of a particular antibody for its particular antigen. However, the variability is not evenly distributed across the entirety of the variable regions. Instead, the variable regions consist of relatively invariant stretches called framework regions (FRs) separated by shorter regions of extreme variability called complementarity determining regions (CDRs) or hypervariable regions. The variable regions of native heavy and light chains each comprise four FRs, largely adopting a beta-sheet configuration, connected by three CDRs, which form loops connecting, and in some cases forming part of, the beta-sheet structure. The CDRs complement an antigen's shape and determine the antibody's affinity and specificity for the antigen. There are six CDRs in both V_{L} and V_{H}. The CDRs in each chain are held together in close proximity by the FRs and, with the CDRs from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)).

The term “hypervariable region” when used herein refers to the amino acid residues of an antibody that are responsible for antigen binding. The hypervariable region generally comprises amino acid residues from a “complementarity determining region” or “CDR” (e.g., around about residues 24-34 (L1), 50-56 (L2) and 89-97 (L3) in the V_{L}, and around about 31-35 (H1), 50-65 (H2) and 95-102 (H3) in the V_{H} when numbered in accordance with the Kabat numbering system; Kabat et al., Sequences of Proteins of Immunological Interest, 5th Ed. Public Health Service, National Institutes of Health, Bethesda, Md. (1991)); and/or those residues from a “hypervariable loop” (e.g., residues 24-34 (L1), 50-56 (L2) and 89-97 (L3) in the V_{L}, and 26-32 (H1), 52-56 (H2) and 95-101 (H3) in the V_{H} when numbered in accordance with the Chothia numbering system; Chothia and Lesk, J. Mol. Biol. 196:901-917 (1987)); and/or those residues from a “hypervariable loop”/CDR (e.g., residues 27-38 (L1), 56-65 (L2) and 105-120 (L3) in the V_{L}, and 27-38 (H1), 56-65 (H2) and 105-120 (H3) in the V_{H} when numbered in accordance with the IMGT numbering system; Lefranc, M. P. et al. Nucl. Acids Res. 27:209-212 (1999), Ruiz, M. et al. Nucl. Acids Res. 28:219-221 (2000)). Optionally the antibody has symmetrical insertions at one or more of the following points 28, 36 (L1), 63, 74-75 (L2) and 123 (L3) in the V_{L}, and 28, 36 (H1), 63, 74-75 (H2) and 123 (H3) in the V_{H} when numbered in accordance with AHo; Honneger, A. and Plunkthun, A. J. Mol. Biol. 309:657-670 (2001)). As used herein, a CDR may refer to CDRs defined by any of these numbering approaches or by a combination of approaches or by other desirable approaches. In addition, a new definition of highly conserved core, boundary and hyper-variable regions can be used.

A “constant region” of an antibody refers to the constant region of the antibody light chain or the constant region of the antibody heavy chain, either alone or in combination. The constant regions of the light chain (C_{L}) and the heavy chain (C_{H}1, C_{H}2 or C_{H}3, or C_{H}4 in the case of IgM and IgE) confer important biological properties such as secretion, transplacental mobility, Fc receptor binding, complement binding, and the like. By convention the numbering of the constant region domains increases as they become more distal from the antigen binding site or amino-terminus of the antibody. The constant regions are not involved directly in binding an antibody to an antigen, but exhibit various effector functions, such as participation of the antibody in antibody dependent cellular cytotoxicity (ADCC), antibody-dependent cellular phagocytosis (ADCP), antibody-dependent neutrophil phagocytosis (ADNP), and antibody-dependent complement deposition (ADCD).

The antibody may be an antibody fragment. “Antibody fragments” comprise only a portion of an intact antibody, generally including an antigen binding site of the intact antibody and thus retaining the ability to bind antigen. Examples of antibody fragments encompassed by the present definition include: (i) the Fab fragment, having V_{L}, C_{L}, V_{H} and C_{H1} domains; (ii) the Fab′ fragment, which is a Fab fragment having one or more cysteine residues at the C-terminus of the C_{H1} domain; (iii) the Fd fragment having V_{H} and C_{H}1 domains; (iv) the Fd′ fragment having V_{H} and C_{H}1 domains and one or more cysteine residues at the C-terminus of the C_{H}1 domain; (v) the Fv fragment having the V_{L} and V_{H} domains of a single antibody; (vi) the dAb fragment which consists of a V_{H} domain; (vii) isolated CDR regions; (viii) F(ab′)₂ fragments, a bivalent fragment including two Fab′ fragments linked by a disulfide bridge at the hinge region; (ix) single chain antibody molecules (e.g. single chain Fv; scFv); (x) “diabodies” with two antigen binding sites, comprising a heavy chain variable domain (V_{H}) connected to a light chain variable domain (V_{L}) in the same polypeptide chain; (xi) “linear antibodies” comprising a pair of tandem Fd segments (V_{H}-C_{H}1-V_{H}-C_{H}1) which, together with complementary light chain polypeptides, form a pair of antigen binding regions.

The antibody may be a chimeric antibody. “Chimeric antibodies” refers to those antibodies wherein one portion of each of the amino acid sequences of heavy and light chains is homologous to corresponding sequences in antibodies derived from a particular species or belonging to a particular class, while the remaining segment of the chains is homologous to corresponding sequences in another. For example, a chimeric antibody may be an antibody comprising antigen binding sequences from a non-human donor grafted to a heterologous non-human, human, or humanized sequence (e.g., framework and/or constant domain sequences). Typically, in these chimeric antibodies, the variable region of both light and heavy chains mimics the variable regions of antibodies derived from one species of mammals, while the constant portions are homologous to the sequences in antibodies derived from another. For example, methods have been developed to replace light and heavy chain constant domains of a monoclonal antibody with analogous domains of human origin, leaving the variable regions of the foreign antibody intact. Alternatively, “fully human” monoclonal antibodies are produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557, incorporated herein by reference). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh) RNA, microRNA (miRNA), small or short interfering (si) RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction.

A nucleic acid encoding a polypeptide often comprises an open reading frame that encodes the polypeptide. Unless otherwise indicated, a particular nucleic acid sequence also includes degenerate codon substitutions.

A “transgene” is used herein to conveniently refer to a nucleic acid sequence/polynucleotide that is intended or has been introduced into a cell or organism. Transgenes include any nucleic acid, such as a gene that encodes an inhibitory RNA or polypeptide or protein, and are generally heterologous with respect to naturally occurring AAV genomic sequences.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, transgene transcribed and the encoded inhibitory RNA or protein expressed. For gene therapy uses and methods, a transduced cell can be in a mammal.

Transgenes under control of inducible promoters are expressed only or to a greater degree, in the presence of an inducing agent, (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting a suitable promoter (constitutive versus inducible; strong versus weak), it is possible to control both the existence and level of expression of a polypeptide in the genetically modified cell. If the gene encoding the polypeptide is under the control of an inducible promoter, delivery of the polypeptide in situ is triggered by exposing the genetically modified cell in situ to conditions for permitting transcription of the polypeptide, e.g., by intraperitoneal injection of specific inducers of the inducible promoters which control transcription of the agent. For example, in situ expression by genetically modified cells of a polypeptide encoded by a gene under the control of the metallothionein promoter, is enhanced by contacting the genetically modified cells with a solution containing the appropriate (i.e., inducing) metal ions in situ.

A nucleic acid/transgene is “operably linked” when it is placed into a functional relationship with another nucleic acid sequence. A nucleic acid/transgene encoding and RNAi or a polypeptide, or a nucleic acid directing expression of a polypeptide may include an inducible promoter, or a tissue-specific promoter for controlling transcription of the encoded polypeptide. A nucleic acid operably linked to an expression control element can also be referred to as an expression cassette.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the invention, such polypeptides encoded by nucleic acid sequences are not required to be identical to the endogenous protein that is defective, or whose activity, function, or expression is insufficient, deficient or absent in a treated mammal.

Non-limiting examples of modifications include one or more nucleotide or amino acid substitutions (e.g., about 1 to about 3, about 3 to about 5, about 5 to about 10, about 10 to about 15, about 15 to about 20, about 20 to about 25, about 25 to about 30, about 30 to about 40, about 40 to about 50, about 50 to about 100, about 100 to about 150, about 150 to about 200, about 200 to about 250, about 250 to about 500, about 500 to about 750, about 750 to about 1000 or more nucleotides or residues).

An example of an amino acid modification is a conservative amino acid substitution or a deletion. In particular embodiments, a modified or variant sequence retains at least part of a function or activity of the unmodified sequence (e.g., wild-type sequence).

Another example of an amino acid modification is a targeting peptide introduced into a capsid protein of a viral particle. Peptides have been identified that target recombinant viral vectors or nanoparticles, to the central nervous system, such as vascular endothelial cells. Thus, for example, endothelial cells lining brain blood vessels can be targeted by the modified recombinant viral particles or nanoparticles.

A recombinant virus so modified may preferentially bind to one type of tissue (e.g., CNS tissue) over another type of tissue (e.g., liver tissue). In certain embodiments, a recombinant virus bearing a modified capsid protein may “target” brain vascular epithelia tissue by binding at level higher than a comparable, unmodified capsid protein. For example, a recombinant virus having a modified capsid protein may bind to brain vascular epithelia tissue at a level 50% to 100% greater than an unmodified recombinant virus.

A “nucleic acid fragment” is a portion of a given nucleic acid molecule. Deoxyribonucleic acid (DNA) in the majority of organisms is the genetic material while ribonucleic acid (RNA) is involved in the transfer of information contained within DNA into proteins. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein. In certain embodiments, the fragment or portion is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this invention, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

The terms “comprising,” “having,” “including,” and “containing” are to be construed as open-ended terms (i.e., meaning “including, but not limited to”) unless otherwise noted.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

### VII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Neuroblastoma Cells Overcome GPC2 CAR T-Cell Killing by Down-Regulating GPC2, but Upregulate NK Cell Ligands

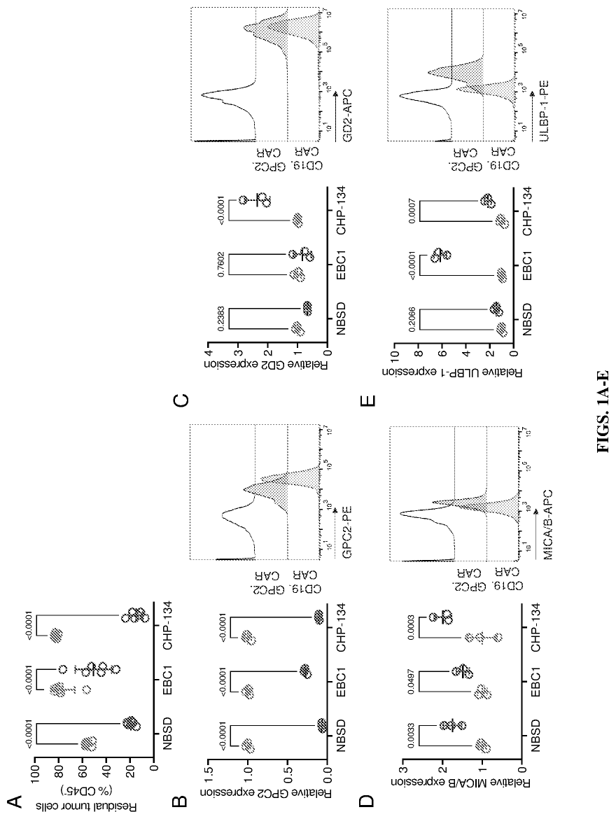

Example 1 provides data addressing antigen modulation in neuroblastoma cells after GPC2 CAR T-cell therapy in vitro. Experiments were performed using 3 different neuroblastoma cells with high GPC2 expression; CHP-134, NB-EBC1 and NBSD. Briefly, neuroblastoma cells were cultured in 6-well plates for 24 h and then treated with GPC2 or CD19 CAR T-cells at an E:T (effector:tumor) ratio of 1:2.5 for 4 days. At that timepoint, tumor cell viability and antigen expression were evaluated by flow cytometry. Neuroblastoma cell viability after GPC2 CAR T-cell treatment was reduced compared to CD19 CAR T-cells (FIG. 1A). However, the residual neuroblastoma cells (CD45-negative) that survived after GPC2 CAR pressure showed significantly reduced GPC2 expression (FIG. 1B), whereas the expression of another targetable tumor antigen (GD2) did not change (FIG. 1C).

Because NK and other innate immune cells have potent antitumor properties and may bypass intrinsic tumor resistant to T-cell killing, the expression of activating NK cell ligands was evaluated in neuroblastoma cells treated with GPC2 CAR T-cells. NK-cell ligands MICA/B and ULBP-1 were upregulated after GPC2 CAR pressure compared to cells treated with CD19 CARs (FIGS. 1D,E). Thus, although neuroblastoma cells downregulate GPC2 to evade CAR T-cell killing, the maintained expression of other targetable tumor antigens such GD2 and the upregulation of activating NK ligands suggest that a CAR T-cell-based GPC2/GD2 dual targeting that exploits antitumor properties of host innate immune cells will be an effective approach to circumvent GPC2 CAR T-cell resistance mechanisms.

### Example 2—Engineering CAR T-Cells to Express GPC2 CAR in the Cell Membrane and to Secrete Bispecific Innate Immune Cell Engagers (BICEs)

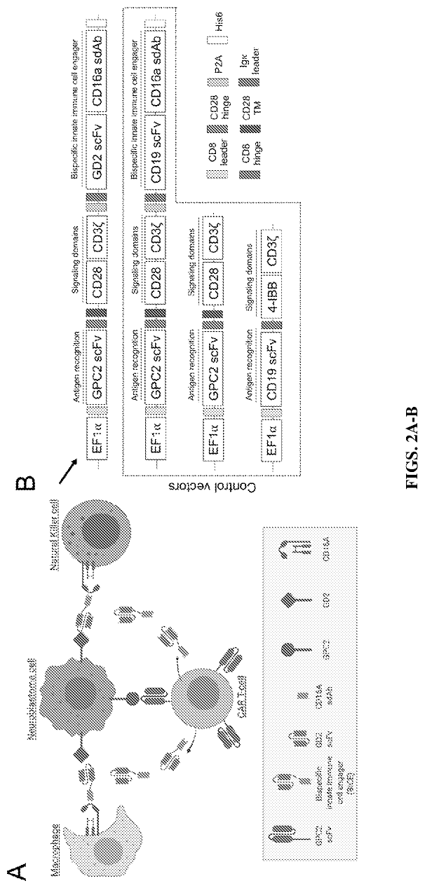

Example 2 provides the rational vector design for CAR T-cells secreting BiCEs. The approach of GPC2 CAR T-cells secreting BiCEs is summarized as a graphical abstract in FIG. 2A. As previously mentioned, BiCEs are composed by tumor-targeted GD2 single-chain variable antibody fragments (scFv) linked to single-domain antibodies (sdAb) targeting CD16A including NK cells and macrophages. The proposed anti-CD16a sdAb have decreased size (15 kDa) compared to conventional CD16 scFvs and thus could result in increased BiCE tissue penetration once delivered by T-cells at the tumor site.

FIG. 2B provides a summarized illustration for bicistronic lentiviral CAR.BiCE vectors. Transduction of human primary T-cells with CAR.BiCEs vectors can lead to i) expression of a CAR against GPC2, ii) a CD28 transmembrane and co-stimulatory domain-based T cell activation and killing against GPC2+ tumors and iii) secretion of the GD2/CD16a-targeted BiCEs. BiCE might be tagged with His-tag to facilitate the detection of the secreted protein in in vitro and in vivo experiments. The preclinical studies to determine the efficacy and safety of the new construct were done in parallel with multiple control vectors: 1) A GPC2 CAR secreting CD19-directed BiCE, 2) a GPC2 CAR alone and 3) a CD19 CAR alone. An additional CD19 CAR secreting GD2 BiCE may be also designed as a non-tumor targeted BiCE-secreting control vector.

### Example 3—Production and Binding Characterization of GD2-Targeted Bispecific Innate Immune Cell Engagers (BiCEs)

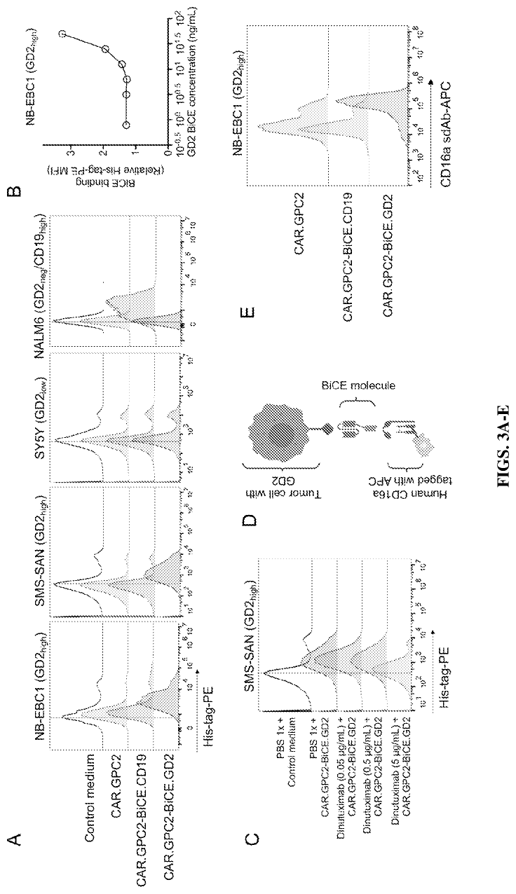

Example 3 provides data on the binding properties of BiCEs. First, to produce BiCEs, HEK 293T cells were transfected with bicistronic constructs and cell supernatants were collected, filtered, and concentrated using Amicon® Ultra-15 Centrifugal Filter Units (nominal molecular weight limit of 10 kD). BiCE concentration was quantified using a competitive His-tag ELISA. To assess whether BiCE bind to GD2-expressing neuroblastoma cells, cSNs were incubated with neuroblastoma cells expressing high (NB-EBC1 and SMS-SAN) or low levels of GD2 (SY5Y) or CD19-expressing leukemia cells (NALM6) for 30 min. Tumor cells were washed, stained with PE-conjugated His-Tag antibodies and analyzed by flow cytometry. His-tag expression was observed in GD2-positive neuroblastoma cells exposed to GPC2.CAR-GD2.BICE cSNs or in leukemia cells exposed to GPC2.CAR-CD19.BICE cSN (FIG. 3A). BiCE binding to GD2-positive NB-EBC1 cells was concentration-dependent, as shown in FIG. 3B. In addition, to corroborate that binding was GD2-specific, a competition assay was performed in which tumor cells were first exposed to FDA-approved anti-GD2 mAb dinutuximab at different concentrations (0.05, 0.5 and 5 μg/mL) and then exposed to GD2 BiCE. Previous exposure to dinutuximab at the highest concentration tested reduced GD2 BiCE binding to NB-EBC1 cells, as shown in FIG. 3C. Thus, these previous studies confirmed that BiCEs bind specifically to tumor cells in a GD2-specific, concentration-dependent manner.

Next, to study whether BiCEs might produce a molecular synapsis between GD2-expressing cells and CD16a, a “sandwich” binding assay was developed as illustrated in FIG. 3D. GD2-expressing NB-EBC1 cells were incubated with cSN from bicistronic vectors and then stained with recombinant human (rh) CD16A protein previously conjugated with APC fluorophore. In this scenario, GD2 single-chain variable fragments (scFvs) bind to tumor cells whereas CD16A single-domain antibody (sdAb) bind to APC-tagged rhCD16A. CD16A sdAb staining was observed in GD2-positive cells exposed to GPC2.CAR-GD2.BiCE cSNs but not in the presence of other constructs (FIG. 3E), thus confirming dual engagement of BiCEs to both GD2 and CD16a epitopes.

### Example 4—Antibody-Dependent Cellular Cytotoxicity (ADCC) and Phagocytosis (ADCP) of GD2-Targeted Bispecific Innate Immune Cell Engagers (BiCEs)

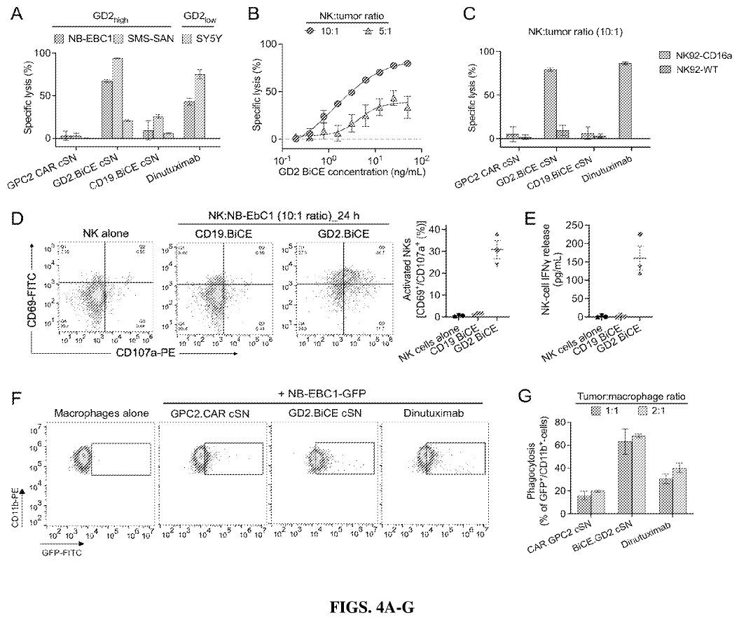

Example 4 provides data on the ADCC and ADCP properties of BiCEs. In a set of experiments, ADCC properties of GD2-targeted BiCEs were evaluated against luciferase-labeled neuroblastoma cell lines with different levels of GD2 using human primary NKs as immune effector cells. To this end, tumor cells were exposed to cSN from bicistronic CAR.BiCE constructs together with primary NKs cells at a NK:tumor ratio of 10:1 for 24 h. Tumor cell viability was determined measuring luciferase signal. FDA-approved dinutuximab (10 μg/mL) was used as positive control for ADCC. NK cell-mediated killing was observed in GD2-expressing cells exposed to GD2 BiCE (5 ng/ml) but not in GD2-negative cells (FIG. 4A). Minimum killing was observed when using CD19-targeted BiCEs at the same concentration. In NB-EBC1 cells, NK cell-mediated killing was dependent on both GD2 BiCE concentration and NK:tumor ratios (FIG. 4B). In a second ADCC experiment, immortalized NK92 cells were used as immune effector cells. NK92 cells resemble characteristics of human NK cells but have low expression of endogenous CD16a, therefore a stable isogenic NK92 cell line with overexpressed CD16a was engineered. NB-EBC1 cells were exposed to BiCEs and then co-cultured with either NK92 wild-type or NK92-CD16a cells for 24 h. As shown in FIG. 4C, only CD16a-expressing NK92 cells induced cytotoxicity when exposed to GD2 BiCEs or dinutuximab. The addition of GD2, but not CD19 BiCE, in co-cultures with primary NK-cells (from 3 different donors) plus GD2⁺ target cells (NB-EbC1) strongly activated NK-cells at 24 h, which displayed strong membrane co-staining of CD69 and CD107a (FIG. 4D) and release of IFN-γ (FIG. 4E). In summary, these studies confirm that GD2-targeted BiCE induce antigen-specific NK cell-mediated killing and activation dependent on engagement of CD16a.

To study whether BiCE might induce ADCP, GFP-labeled NB-EBC1 cells (GD2-high) were exposed to cSN from bicistronic constructs (BiCE concentration of 5 ng/mL) together with human monocyte-differentiated macrophages at 2 different ratios. Phagocytosis was measured by quantifying GFP/CD11b-positive macrophages by flow cytometry, as shown in FIG. 4F. FDA-approved dinutuximab (10 μg/mL) was used as positive control. Percentage of macrophages engulfing NB-EBC1 cells was higher in GD2 BiCEs compared to both GPC2.CAR cSN or dinutuximab (FIG. 4G). In conclusion, this ADCP study confirm that GD2 BiCEs induce macrophage-driven tumor cell phagocytosis in vitro.

### Example 5—Human Primary T Cells Transduced with Bicistronic Vectors Express GPC2 CAR on the Surface that Induces GPC2-Dependent Tumor Killing and T-Cell Activation

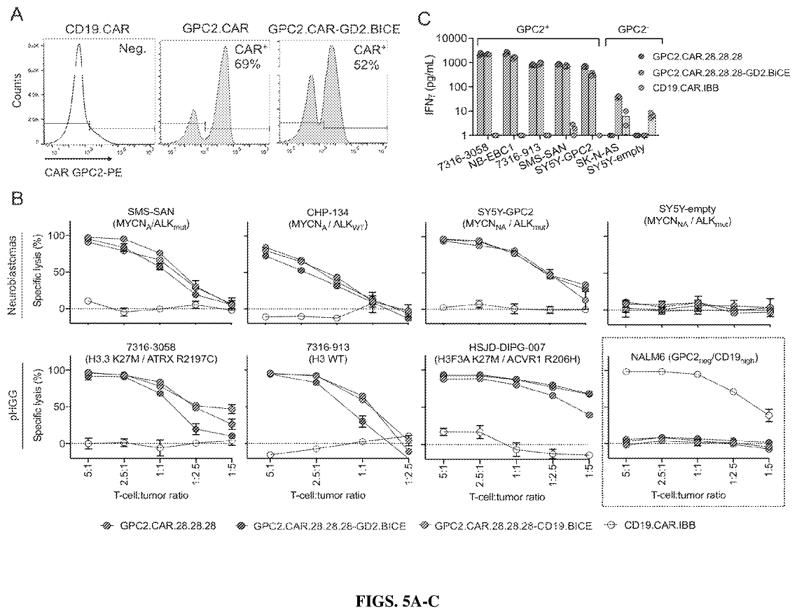

Example 5 provides data on cytotoxic properties of T cells engineered with bicistronic constructs. Primary human T-cells were obtained from healthy donors at University of Pennsylvania under informed consent, activated with CD28/CD3 stimulatory beads and transduced with different bicistronic vectors. T-cells were expanded during 14 days in the presence of IL-15 and IL-7 cytokines. At the end of expansion, surface expression of GPC2 CAR in T cells was determined by flow cytometry using recombinant human GPC2 protein tagged with PE. Percentage of CAR positive cells is indicated in FIG. 5A. To determine if GPC2.CAR-GD2.BICE T-cells retain cytotoxic properties by targeting GPC2 in vitro, luciferase-labeled GPC2-expressing neuroblastoma and high-grade glioma (HGG) cells were co-cultured with transduced T cells at different E:T ratios for 24 h. Tumor killing was evaluated measuring tumor-derived luciferase signal. GPC2-negative NALM6 cells (leukemia) were used as controls. As shown in FIG. 5B, GPC2.CAR-GD2.BiCE T-cells showed dose-dependent cytotoxicity in both neuroblastoma and HGG cell lines but not in GPC2-negative leukemia cells (FIG. 5B, dotted frame). T-cell activation was evaluated by measuring IFN-γ in supernatants from killing assays (E:T ratio of 2.5:1). As indicated in FIG. 5C, both GPC2 CAR or GPC2.CAR-GD2.BICE T cells secreted IFN-γ at similar levels when exposed to GPC2-expressing cells but not in antigen-negative cells. In conclusion, human primary T cells transduced with bicistronic CAR.BiCE vectors maintain comparable GPC2 CAR-based cytotoxic properties to first-generation CAR T-cells by targeting GPC2.

### Example 6—Human Primary T-Cells Transduced with Bicistronic Vectors Secret BiCEs and Activate Primary NK Cells to Kill GD2-Expressing Cells

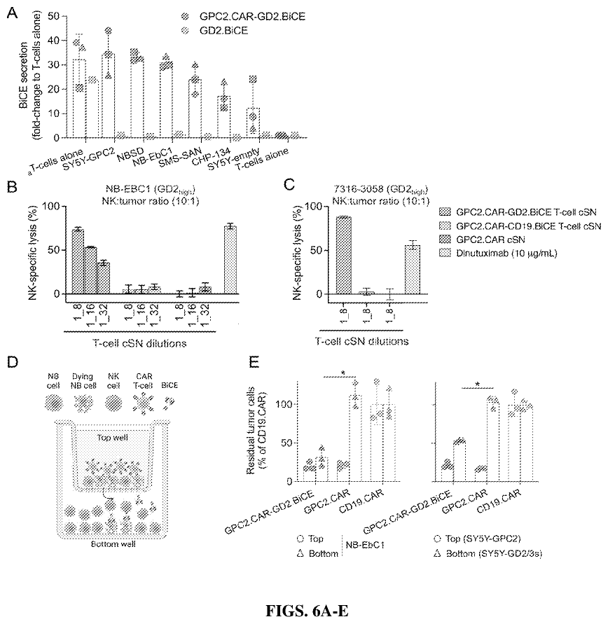

Example 6 provides data on BiCE secretion and bystander activation of NK cells by the engineered T-cells. T-cell secretion of GD2 BiCE was measured by ELISA in T-cells alone, activated with stimulatory beads (aT-cells) or after exposure to target cells with different levels of GPC2. As shown in FIG. 6A, GPC2 CAR T-cell secretion of GD2 BiCE was markedly increased after exposure with either beads or antigen-positive cells. In contrast, T-cells transduced with GD2 BiCE vector (without the GPC2 CAR region) showed enhanced secretion after beads but not when exposed to neuroblastoma cells (FIG. 6A), further confirming T-cell activation-dependent BiCE release. Next, to determine if T-cell-secreted BICE could induce NK cell-mediated neuroblastoma cell killing in vitro, luciferase-labeled NB-EBC1 cells were exposed to different dilutions of T-cell cSNs together with primary human NK cells at a E:T ratio of 10:1 for 24 h. As indicated in FIG. 6B, T-cell-secreted GD2 BiCEs induced NK cell-mediated killing in a dose-dependent manner but not those from control vectors and NK cytotoxicity was comparable to that induced by dinutuximab at 10 μg/mL. NK cell-mediated killing was also appreciated when GPC2.CAR-GD2.BiCE T-cell supernatants were added to GD2-expressing high-grade glioma cells (FIG. 6C). Finally, we evaluated whether GPC2.CAR-GD2.BICE T-cells could induce bystander NK activation in Transwell assays, in which CAR T-cells were plated with neuroblastoma cells in the top chambers and NK cells together with neuroblastoma cells in the bottom chambers (FIG. 6D). As shown in FIG. 6E, both GPC2.CAR or GPC2.CAR-GD2.BICE T-cells killed GPC2+ neuroblastoma cells in the top chambers, whereas only GPC2.CAR-GD2.BiCE T-cells were able to efficiently activate NK cells in the bottom chambers to kill either GPC2⁺/GD2⁺ (NB-EBC1) or GPC2⁻/GD2⁺ (SY5Y) cells. In summary, BiCE secreted by activated T-cells target GD2-expressing neuroblastoma and HGG cells and induce strong bystander NK cell-mediated killing.

### Example 7—In Mice, Human T-Cells Transduced with Bicistronic Vectors Locally Release GD2 BiCE which Enhances Accumulation of NK Cells in the Tumor Bed

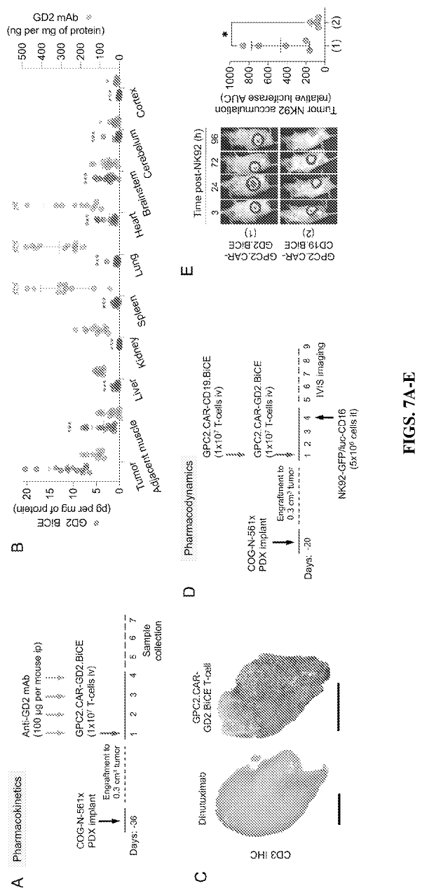

Example 7 provides data on the pharmacokinetics (PK) and pharmacodynamics (PD) of GD2 BiCE after in vivo administration of engineered T-cells in mice bearing neuroblastoma PDXs. FIG. 7A shows the experimental design utilized to study the PK of GD2 BiCE after one intravenous dose of 10 million T-cells compared to GD2 mAbs after 4 consecutive intraperitoneal doses of dinutuximab. As shown in FIG. 7B, GD2 BiCEs were detected in the tumor but not in mouse healthy tissues, whereas anti-GD2 mAbs were detected at high concentrations in tumor, liver, kidney, spleen, lung and heart, overall suggesting a GPC2-targeted delivery of GD2 BiCE selectively by activated T-cells. As expected, robust intratumor infiltration of T-cells was confirmed in CAR.BiCE T-cell-treated animals (FIG. 7C). For PD studies, neuroblastoma PDX-bearing animals were intravenously administered with either 10 million GPC2.CAR-GD2.BiCE or GPC2.CAR-CD19.BICE T-cells, followed by intratumor injection of luciferase-labelled CD16-overexpressing NK92 cells 4 days later (FIG. 7D). NK92-cell accumulation was monitored by IVIS imaging at different timepoints after injection (FIG. 7D). Intratumor NK92 luciferase signal was prolonged for longer periods of time (up to 96 h) in animals previously treated with GPC2.CAR-GD2.BICE but not in those treated with GPC2.CAR-CD19.BiCE T-cells (FIG. 7E). In summary, PK and PD studies demonstrate that GPC2.CAR-GD2.BiCE T-cells locally release BiCE in the tumor parenchyma and promote NK cell accumulation in the tumor.

### Example 8—GPC2.CAR-GD2.BICE T-Cells are Effective In Vivo Against Neuroblastoma PDXs Reconstituted with Donor-Matched PBMCs

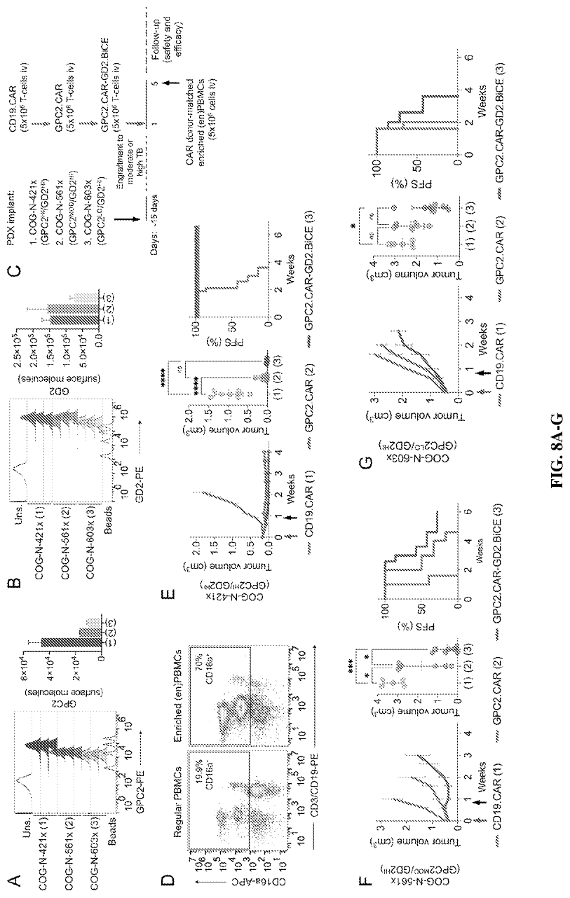

Example 8 provides data on the in vivo efficacy of GPC2.CAR-GD2.BiCE T-cells. The antitumor activity of engineered T-cells was tested in three different neuroblastoma PDXs expressing different levels of GPC2 and GD2 (COG-N-421x, COG-N-561x and COG-N-603x) (FIGS. 8A-B). Briefly, immunodeficient NSG mice bearing 0.1-0.5 cm³ PDX tumors were intravenously injected with 5 million CD19.CAR, GPC2.CAR or GPC2.CAR-GD2.BiCE T-cells on day 0 (n=5-6 mice per group) followed by 5 million donor-matched innate immune cells, as summarized in FIG. 8C. To obtain innate immune cells, T- and B-cells were depleted using CD3 and CD19 beads respectively, achieving 70% of cells expressing CD16a that might be engaged by T-cell secreted BiCEs (FIG. 8D). In the PDX model with high GPC2 and GD2 expression (COG-N-421x), both GPC2.CAR and GPC2.CAR-GD2.BiCE T-cells displayed robust tumor control compared to CD19.CARs that was prolonged up to 4 weeks after T-cell injection (FIG. 8E). Most importantly, in PDX models with medium and low GPC2 but high GD2 expression (COG-N-561x and COG-N-603x, respectively), GPC2.CAR-GD2.BiCE T-cells displayed increased antitumor activity compared to GPC2.CARs alone (FIGS. 8F-G), which validates the additional antitumor effect mediated by the GD2 BiCE-engaged innate immune cells. Thus, the in vivo studies validate the antitumor efficacy of GPC2 CAR T-cell secreting GD2 BiCEs against clinically relevant, antigen heterogeneous neuroblastoma PDX models and such efficacy is significantly higher than GPC2 CAR T cells alone.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
1. A. L. Yu et al., Anti-GD2 antibody with GM-CSF, interleukin-2, and isotretinoin for neuroblastoma. N Engl J Med 363, 1324-1334 (2010).
2. S. L. Maude et al., Tisagenlecleucel in Children and Young Adults with B-Cell Lymphoblastic Leukemia. N Engl J Med 378, 439-448 (2018).
3. C. F. Malone, K. Stegmaier, Scratching the Surface of Immunotherapeutic Targets in Neuroblastoma. Cancer Cell 32, 271-273 (2017).
4. K. R. Bosse et al., Identification of GPC2 as an Oncoprotein and Candidate Immunotherapeutic Target in High-Risk Neuroblastoma. Cancer Cell 32, 295-309 e212 (2017).

1. A polynucleotide having a first coding sequence that encodes a chimeric antigen receptor (CAR) comprising, from 5′ to 3′: (i) an ectodomain comprising a single chain antibody variable region that binds selectively to Glypican 2 (GPC2), (ii) a transmembrane domain, and (iii) an endodomain, wherein the endodomain comprises a signal transduction function when the single-chain antibody variable region is engaged with Glypican 2; and a second coding sequence that encodes a fusion protein comprising: (i) a single chain antibody variable region that binds selectively to GD2; and (ii) a single domain antibody (sdAb) that binds CD16A.

2. The polynucleotide of claim 1, wherein the CAR comprises a flexible hinge positioned between the ectodomain and the transmembrane domain.

3. The polynucleotide of claim 2, wherein the flexible hinge is a CD28 hinge.

4. The polynucleotide of claim 2, wherein the flexible hinge is a CD28 hinge having the sequence of SEQ ID NO: 6.

5. The polynucleotide of claim 1, wherein the transmembrane domain is a CD28 transmembrane domain.

6. The polynucleotide of claim 5, wherein the transmembrane domain is a CD28 transmembrane domain having the sequence of SEQ ID NO: 7.

7. The polynucleotide of claim 1, wherein the endodomain comprises a CD28 co-stimulatory domain.

8. The polynucleotide of claim 7, wherein the endodomain comprises a CD28 co-stimulatory domain having the sequence of SEQ ID NO: 8.

9. The polynucleotide of claim 1, wherein the endodomain comprises a 4-IBB co-stimulatory domain.

10. The polynucleotide of claim 9, wherein the endodomain comprises a 4-IBB co-stimulatory domain having the sequence of SEQ ID NO: 9.

11. The polynucleotide of claim 1, wherein the endodomain comprises a CD3zeta co-stimulatory domain.

12. The polynucleotide of claim 1, wherein the endodomain comprises a CD3zeta co-stimulatory domain having the sequence of SEQ ID NO: 10.

13. The polynucleotide of claim 1, wherein the GPC2 single chain antibody is encoded by heavy and light chain variable sequences of SEQ ID NOS: 3 and 4, respectively.

14. The polynucleotide of claim 1, wherein the GPC2 single chain antibody is encoded by heavy and light chain variable sequences having at least 70%, 80%, 90%, or 95% identity to SEQ ID NOS: 3 and 4, respectively.

15. The polynucleotide of claim 1, wherein the GPC2 single chain antibody comprises heavy and light chain variable sequences of SEQ ID NOS: 1 and 2, respectively.

16. The polynucleotide of claim 1, wherein the GPC2 single chain antibody comprises heavy and light chain variable sequences having 95% identity to SEQ ID NOS: 1 and 2, respectively.

17. The polynucleotide of claim 1, wherein the second coding region is a bipecific innate immune cell engager (BiCE) that comprises a GD2 single chain antibody variable region fused to a CD16A single domain antibody.

18. The polynucleotide of claim 1, wherein the GD2 single chain antibody comprises heavy and light chain variable sequences of SEQ ID NOS: 12 and 13, respectively.

19. The polynucleotide of claim 1, wherein the CD16A single domain antibody is characterized by CDR sequences SEQ ID NOS: 16-18.

20. The polynucleotide of claim 1, wherein the CD16A single domain antibody comprises the sequence of SEQ ID NO: 15.

21. The polynucleotide of claim 1, wherein the CD16A single domain antibody comprises a sequence having 95% identity to SEQ ID NO: 15.

22. The polynucleotide of claim 1, further comprising a sequence encoding a CD8 leader sequence positioned 5′ of the first coding sequence.

23. The polynucleotide of claim 1, further comprising a sequence encoding a cleavable peptide positioned between the first coding sequence and the second coding sequence.

24. The polynucleotide of claim 23, wherein the cleavable peptide is P2A.

25. The polynucleotide of claim 1, further comprising a sequence encoding a Igκ leader sequence positioned 5′ of the second coding sequence.

26. The polynucleotide of claim 1, further comprising a His6 sequence positioned 3′ of the second coding sequence.

27. The polynucleotide of claim 1, further comprising a promoter sequence positioned 5′ of the first coding sequence.

28. The polynucleotide of claim 27, wherein the promoter is a constitutive promoter.

29. The polynucleotide of claim 27, wherein the promoter is an EF1α promoter.

30. An expression vector comprising the polynucleotide of claim 1.

31. A viral vector comprising the polynucleotide of claim 1.

32. The viral vector of claim 31, wherein the viral vector is a lentiviral vector.

33. A cell comprising the polynucleotide of claim 1.

34. The cell of claim 33, wherein the polynucleotide is integrated into the genome of the cell.

35. The cell of claim 33, wherein the cell is a T cell.

36. The cell of claim 35, wherein the T cell expresses the chimeric antigen receptor on its surface.

37. The cell of claim 35, wherein the T cell secretes the fusion protein.

38. A composition comprising the cell of claim 33, the fusion protein encoded by the second coding sequence, and a pharmaceutically acceptable carrier.

39. The composition of claim 38, wherein the composition further comprises an additional active agent.

40. A method of treating cancer in a patient in need thereof, the method comprising administering to the patient an effective amount of the cell of claim 33.

41. The method of claim 40, wherein the cancer is a solid tumor.

42. The method of claim 40, wherein the cancer is a neuroblastoma or glioma.

43. The method of claim 40, wherein the patient is a pediatric patient.

44. The method of claim 40, wherein the cells of the cancer express GPC2 on their surface.

45. The method of claim 40, wherein the cells of the cancer express GD2 on their surface.

46. The method of claim 40, wherein the method activates the patient's innate immune effector cells to target the cancer.

47. The method of claim 40, wherein the method induces antibody-dependent cellular cytotoxicity against the cancer.

48. The method of claim 40, wherein the method induces antibody-dependent cellular phagocytosis against the cancer.

49. The method of claim 40, wherein the cells are allogeneic or autologous to the patient.

50. The method of claim 40, wherein the cells or the composition are administered systemically.

51. The method of claim 40, further comprising administering a second anti-cancer therapy to the patient.

52. The method of claim 51, wherein the second anti-cancer therapy comprises a comprises a surgical therapy, chemotherapy, radiation therapy, cryotherapy, hormone therapy, immunotherapy, or cytokine therapy.
